SEQUENCE LISTING (15 sequences)

SEQ ID NO: 1 - protein, 4 aa, Drosophilia
  misc_feature (1)..(1): Xaa is Lys or Arg
XTQT

SEQ ID NO: 2 - protein, 10 aa, Drosophilia
SYSKETQTPL

SEQ ID NO: 3 - protein, 5 aa, Drosophilia
  misc_feature (1)..(1): Xaa is Lys, His or Arg
XXTQT

SEQ ID NO: 4 - protein, 5 aa, Drosophilia
KETQT

SEQ ID NO: 5 - protein, 5 aa, Drosophilia
KSTQT

SEQ ID NO: 6 - protein, 5 aa, Drosophilia
KGTQT

SEQ ID NO: 7 - protein, 5 aa, Drosophilia
RSTQT

SEQ ID NO: 8 - protein, 5 aa, Drosophilia
KATQT

SEQ ID NO: 9 - protein, 17 aa, Drosophilia
MKDTGIQVDRDLDGKSH

SEQ ID NO: 10 - protein, 26 aa, Drosophilia
CFITKALGISYGRKKRRQRRRPPQGS

SEQ ID NO: 11 - protein, 12 aa, Drosophilia
CMHIESLDSYTC

SEQ ID NO: 12 - protein, 12 aa, Drosophilia
CMYIEALDKYAC

SEQ ID NO: 13 - protein, 17 aa, Drosophilia
EAALAEALAEALEALAA

SEQ ID NO: 14 - protein, 10 aa, Drosophilia
CSYSKETQPL

SEQ ID NO: 15 - protein, 4 aa, Drosophilia
GGGS

(19) United States
(12) Patent Application Publication
(10) Pub. No.: US 2005/0053591 A1
(43) Pub. Date: Mar. 10, 2005

(54) Compositions and uses of motor protein-binding moieties

(75) Inventor: Suzie Hwang Pun, Torrance, CA (US)

Correspondence Address: ROPES & GRAY LLP, ONE INTERNATIONAL PLACE, BOSTON, MA, 02110-2624, US

(73) Assignee: Insert Therapeutics, Inc., Pasadena, CA (US)

(21) Appl. No.: 10/637,898

(22) Filed: Aug. 8, 2003

Related U.S. Application Data
(60) Provisional application No. 60/402,229, filed on Aug. 8, 2002.

(51) Int. Cl. A61K 38/43; C12N 9/00

(52) U.S. Cl. 424/94.1; 435/183

(57) ABSTRACT

The invention provides methods and reagents for efficient transport of macromolecules (such as drug therapeutics including nucleic acids and polypeptides) linked to motor protein-binding moieties (MPBM) intracellularly to a specific subcellular localization. One exemplary MPBP is dynein binding moiety (DBM). Drug therapeutics linked to DBM can be selectively, specifically, and actively transported intracellularly to the nucleus or peri-nuclear region, thus enhancing the uptake of such drug therapeutics into the target subcellular localization.

### REFERENCE TO RELATED APPLICATIONS

This application claims the benefit of earlier filing date, under 35 U.S.C. 119(e), of U.S. Provisional Application No. 60/402,229, filed on Aug. 8, 2002, the entire content of which is incorporated herein by reference.

### BACKGROUND OF THE INVENTION

Many therapeutics need to be directed to specific organelles or subcellular locations within the cell for maximum effect. For example, drugs for lysosomal storage disease may need to be directed to lysosomes, while gene delivery agents generally require nuclear entry in order for gene transcription to occur. One major barrier in gene delivery therefore is the nuclear membrane, as evidenced by the dependence of transfection on cell division. Brunner et al. (2000) Gene Ther 7:401-407. A well-studied approach to nuclear delivery involves the use of nuclear localization signals (NLS) to facilitate passage through nuclear pores. Richardson et al. (1986) Cell 44:79; Subramanian et al. (1999) Nat Biotech 17:873-877; and Zanta et al. (1999) PNAS 96:91-96. These are naturally occurring peptide sequences used to direct proteins to the nucleus. Another approach for gene delivery utilizes specific sequences in the delivered nucleic acid that mediate nuclear delivery, possibly by being recognition sequences for proteins that contain NLSs themselves. Wilson et al. (1999) J Biol Chem 274:22025-22032; Dean et al. (1997) Experimental Cell Res 230:293-302. While NLSs work sufficiently for smaller molecules such as proteins or short DNA sequences, a recent review concludes that in general these approaches do not significantly enhance delivery of larger molecules. Wilson et al. supra; Dean et al. supra; Chan et al. (2000) Gene Ther 7:1690-1697; Chan et al. (1999) Human Gene Therapy 10:1695-1702. One possible explanation for this phenomenon is that these larger molecules still do not have access to the nuclear membrane due to limited diffusional mobility in the cytoplasm. Lukacs et al. determined the diffusion coefficients of DNA in the cell cytoplasm (D_{cyto}) by spot photobleaching of cells and compared the values to the diffusion coefficients of DNA in water (D_{w}). (Lukacs et al. (2000) J Biol Chem 275:1625-1629). The D_{cyto}/D_{w} is about 0.19 for a 100 base pair DNA molecule. However, D_{cyto}/D_{w} dropped dramatically to less than 0.01 for 2000 bp DNA, and even less for larger macromolecules. For example, most plasmids containing therapeutic genes are at least 5000 bp in size. This indicates that diffusion coefficients in the cytoplasm are lower than in water, and they depend on or are affected by, among other things, the size of the macromolecule (such as DNA) to be delivered intracellularly, the viscosity of cytoplasm, the increased diffusion path resulting from crowding and collisions with intracellular compartments, and the non-specific binding with cytoskeletal structures or organelles.

Therefore, efficient intracellular delivery of certain macromolecule drug therapeutics may require active (energy-dependent) transport through the cytoplasm to the desired organelle, granule, subcellular compartment, or membrane fraction rather than passive diffusion.

### SUMMARY OF THE INVENTION

One aspect of the invention provides a motor protein therapeutic represented by one of the general formulas:
A-L-B (I)
or
A::B (II)
wherein
A represents a moiety which binds to a motor protein;
B represents a drug moiety which produces a change in the growth state or phenotype of a cell, or a change in concentrations or rates of protein internalization, processing, or excretion, in a manner dependent on or enhanced by a particular subcellular localization of B;
L represents a bond or a linker group which covalently attaches A and B, and
:: represents a non-covalent interaction between A and B.

In one embodiment, the motor protein is a conventional kinesin (kinesin 1), a heterotrimeric kinesin 11, a homodimeric kinesin 11, an Unc104/KIF1 protein, or a myosin V.

In one embodiment, the motor protein is a dynein, such as a cytoplasmic dynein or an axonemal dynein. In a preferred embodiment, the motor protein is a cytoplasmic dynein, and wherein moiety A binds to a subunit of the cytoplasmic dynein.

In one embodiment, the subunit is a dynein light chain (DLC), for example, DLC8.

In one embodiment, the moiety A comprises a polypeptide or peptidomimetic with an amino acid sequence consisting essentially of SEQ ID NO: 1, or SEQ ID NO: 1.

In one embodiment, the moiety A comprises a polypeptide or peptidomimetic with an amino acid sequence consisting essentially of SEQ ID NOs: 2, 4, 5, 6, 7, or 8, or SEQ ID NOs: 2, 4, 5, 6, 7, or 8.

In one embodiment, the moiety A comprises a polypeptide or peptidomimetic with an amino acid sequence consisting essentially of Xaa1-Xaa2-Thr-Gln-Thr (SEQ ID NO: 3) or SEQ ID NO: 3,
wherein,
Xaa1 represents an amino acid residue with a positively charged sidechain,
Xaa2 represents an amino acid residue with a polar or a neutral sidechain.

In one embodiment, the Xaa1 is Lys, His or Arg.

In one embodiment, the Xaa2 is a polar sidechain amino acid selected from Arg, Asn, Asp, Cys, Glu, Gln, His, Lys, Ser or Thr.

In one embodiment, the Xaa2 is a neutral sidechain amino acid selected from Ala, Asn, Cys, Gln, Gly, His, Ile, Leu, Met, Phe, Pro, Ser, Thr, Trp, Tyr or Val.

In one embodiment, the Xaa2 is a small neutral sidechain amino acid selected from Gly, Ala or Ser.

In one embodiment, the Xaa2 is a negative polar sidechain amino acid selected from Asp or Glu.

In one embodiment, the Xaa2 is Ala, Gly, Glu or Ser.

In one embodiment, the moiety A is a DLC8-binding domain peptide sequence from neuronal nitric-oxide synthase (nNOS), proapoptotic Bcl-2 family protein Bim, Drosophilia mRNA localization protein Swallow, transcriptional regulator IκB, or postsynaptic scaffold protein GKAP.

In one embodiment, the moiety A is a DLC8-binding domain of nNOS (neuronal nitric-oxide synthase) represented by SEQ ID NO: 9.

In one embodiment, the moiety A is a small organic molecule which selectively binds to DLC8.

In one embodiment, the moiety A binds to cytoplasmic dynein with a dissociation constant K_{d} of no more than 10⁻⁴M.

In one embodiment, the moiety A comprises two or more repeats of a polypeptide which binds to the motor protein.

In one embodiment, the moiety A comprises a peptidomimetic of a polypeptide which binds to the motor protein.

In one embodiment, the moiety A comprises a small organic molecule.

In one embodiment, the drug moiety B is a nucleic acid.

In one embodiment, the nucleic acid is an oligonucleotide, an anti-sense oligonucleotide, an siRNA, or a plasmid.

In one embodiment, the drug moiety B is a polypeptide or a peptidomimetic thereof.

In one embodiment, the polypeptide is a transcriptional regulator, an inducer or inhibitor of programmed cell death, or an intrabody (functional antibody) with intracellular targets.

In one embodiment, the drug moiety B is a microsphere, a liposome, a small organic molecule, or a large synthetic molecule.

In one embodiment, the drug moiety B interacts with a nuclear target.

In one embodiment, the nuclear target is a transcription factor, a histone, or a protein or protein complex which interacts with DNA and regulate gene expression or chromatin structure, a nuclear hormone or steroid receptor, a histone acetylase or deacetylase, a DNA methyltransferase, an enzyme which covalently modifies DNA, a kinase, a phosphatase, a protease, a lipase, an RNA polymerase, a DNA polymerase, a DNA primase, a DNA topoisomerase, a DNA helicase, a nuclease, or an ATPase.

In one embodiment, the drug moiety B is an inhibitor or activator of the nuclear target.

In one embodiment, either A or B or both include functionalities for enhancing cellular uptake and/or transmembrane movement.

In one embodiment, either A or B or both includes groups which can be cleaved (hydrolyzed, reduced, or other means in the art) to form an active drug moiety B not linked to A.

In one embodiment, the subcellular localization is nucleus.

In one embodiment, the subcellular localization is lysosome, mitochondria, ER (Endoplasmic Reticulum), Golgi complex, or a membrane fraction.

In one embodiment, A and B are covalently linked by L through chemical cross-linking.

In one embodiment, L is an amino acid or a polypeptide.

In one embodiment, L includes a bond that can be cleaved (hydrolyzed, reduced, or other means known in the art) or enzymatically degraded under physiological condition once at the subcellular localization.

In one embodiment, the non-covalent interaction :: is ionic interaction, hydrogen bond, van der Waals interaction, hydrophobic interaction, or simultaneous binding of A and B to a third molecule.

In one embodiment, the non-covalent interaction :: is direct binding between A and B.

In one embodiment, the direct binding between A and B is mediated by a pair of heterologous interacting polypeptides.

In one embodiment, the pair of heterologous interacting polypeptides are biotin and streptavidin or avidin.

In one embodiment, the non-covalent interaction becomes unstable under physiological condition once at the subcellular localization.

In one embodiment, the amino acid is Cysteine.

In one embodiment, the polypeptide comprises a terminal Cysteine and a spacer polypeptide.

In one embodiment, the spacer polypeptide comprises one or more repeats of Gly-Gly-Gly-Ser (SEQ ID NO: 15).

It should be understood that any of the above described embodiments can be combined when appropriate.

Another aspect of the invention provides a nucleic acid encoding a proteinaceous motor protein therapeutic of any of the above embodiments, or the complement nucleic acid thereof.

Another aspect of the invention provides a vector comprising any of the above suitable nucleic acids.

Another aspect of the invention provides a host cell comprising any of the vectors or the nucleic acids described above.

Another aspect of the invention provides a method of delivering a drug moiety B to a particular subcellular localization of a cell, comprising contacting the cell with any of the above embodiments of motor protein therapeutics.

### BRIEF DESCRIPTION OF THE DRAWINGS

FIG. 1 The dynein-binding peptide facilitates transport toward the nucleus. In several cells (white arrows), plasmid DNA accumulates around the nuclear membrane.

### DESCRIPTION OF THE INVENTION

I. Overview

The cell contains several families of motor proteins (e.g. myosin V, kinesins and dyneins). These proteins are able to translate chemical energy from ATP hydrolysis to mechanical force or motion. Generally, these proteins are associated with a cytoskeletal structure or filament, using these structures as a scaffold. Myosin proteins transports along actin filaments while kinesin and dynein proteins traverse respectively toward the plus and minus ends of microtubules. These proteins have recognition sequences for their “cargo.”

Cytoplasmic dynein is a microtubule-based molecular motor that has been implicated in a wide variety of functions including retrograde organelle movement, nuclear migration, mitotic spindle alignment, and axonal transport in eukaryotic cells (Holzbaur and Vallee, Annu Rev Cell Biol. 10: 339-72, 1994; Hirokawa, Science 279, 519-526). It moves along a tubulin polymer through repetitive binding and release cycles that are tightly coordinated with force generation and nucleotide hydrolysis (Johnson, 1985). The enzyme is a multisubunit complex assembly containing two molecules of Heavy Chains (DHC, ˜530 kDa), several Intermediate Chains (DIC, ˜74 kDa) and Light Intermediate Chains (DLIC, 53-59 kDa), and a number of Light Chains (DLC, 8-22 kDa). It has previously been demonstrated that dynein travels along the microtubule highways toward the nuclei of the cells by using ATP hydrolysis to trigger conformational changes in the protein. Dynein therefore helps to transport proteins and organelles in the retrograde direction (toward the nuclei). See Hirokawa, N. (1998) Science 279:519-526. The light chain protein, DLC8, is a highly conserved light chain of the dynein complex, which has been shown to interact with several diverse cellular targets. For a detailed review of the structure of dynein, see King, Biochim. Biophy. Acta 1496: 60-75, 2000.

The present invention is directed to the use of motor protein-binding moieties (or “MPBMs”), such as dynein-binding moieties (or “DBMs”), e.g., agents that bind to a motor protein component (for example, dynein light chains such as DLC8), to direct the subcellular (such as nuclear) transport and/or localization of an associated agent. In certain preferred embodiments, the subject conjugates are represented by the general formula (I):
A-L-B (I)
wherein A represents a motor protein-binding moiety; B represents a drug or drug delivery moiety which binds to and/or alters activity of one or more subcellular targets (protein, DNA, etc); and L represents a bond or a linker group which covalently attaches A and B.

In addition to covalently attaching A and B, the linker can also be selected based on its ability to decrease steric interference between the moieties. In certain embodiments, the linker includes a bond that can be cleaved or enzymatically degraded under physiological conditions, e.g., in the nucleus of a cell.

In other embodiments, the subject conjugates are represented by the general formula (II):
A::B (II)
wherein A represents a motor protein-binding moiety; B which represents a drug moiety which binds to and/or alters activity of one or more subcellular targets (protein, DNA, etc); and :: represents a non-covalent interaction between A and B, such as an ionic interaction, or any non-covalent force that stably associates A and B. For example, in the latter case, :: can be a third protein that simultaneously and stably binds A and B such that A and B are stably associated.

In certain embodiments, the non-covalent interaction :: may become unstable under physiological conditions once at the target subcellular localization (such as inside the nucleus or endosome), thus releasing A from B.

The motor protein-binding moiety (such as DBM) can include one or more “motor protein binding sequences,” each of which is independently capable of binding to at least a subunit of a motor protein.

As described in further detail, the motor protein-binding moiety can be, for example, a peptide, a peptidomimetic, a small organic molecule, or a large synthetic molecule.

The subject compositions can be used to enhance the activity of a wide range of molecules which affect cellular function by acting on organelle targets in a desired subcellular localization (such as the nucleus) of cells. Such agents, B, include peptides, peptidomimetics, nucleic acids, small organic molecules, or large synthetic molecules.

Merely to illustrate, B can be an agent that interacts with a transcription factor, a histone, an enzyme specifically localized within an organelle, or other protein or protein complex which interact with DNA and regulate gene expression or chromatin structure. Such targets can include cytoplasmic enzymes, nuclear hormone/steroid receptors (such as receptors for glucocorticoids, mineralocorticoids, sex hormones or ecdysone), histone acetylases or deacetylases, DNA methyltransferases and other enzymes which covalently modify DNA, kinases (such as cyclin dependent kinases), phosphatases (such as cdc25 phosphatases), proteases, lipases, RNA polymerases, DNA polymerases, DNA primases, DNA topoisomerases, DNA helicases, nucleases, ATPases (such as chromatin remodeling ATPases), and the like. The agent can be an inhibitor of an intrinsic enzymatic activity, an inhibitor (or potentiator) of protein-protein, protein-DNA and/or protein-lipid interactions, an intercalating agents (including fluorescent dyes) or the like. In some cases, the agent can be a nucleic acid, such as a decoy sequence which binds to a transcription factor or repressor, an antisense sequence, or a double stranded RNA interference construct. The subject invention also contemplates that the agent can be a molecule which interacts with and alters the structure changes of the nuclear envelope.

Either or both of A and B can include other functionalities, such as functionalities for enhancing cellular uptake (across the cell membrane and into a cytoplasmic compartment), membrane translocation (such as entering or exiting a specific compartment of an organelle, etc.), or groups which can be cleaved (hydrolyzed, reduced, or other means known in the art) to form the active drug (e.g., the drug moiety of the motor protein binding therapeutic is a prodrug).

The subject conjugates can be synthesized chemically. For those embodiments where the entire conjugate is a peptide or polypeptide, the conjugate can be produced recombinantly. Accordingly, the subject invention also contemplates coding sequences for certain of the subject motor protein therapeutics, vectors encompassing such coding sequences, or host cells encompassing such vectors.

The subject conjugates may be transported actively to a variety of subcellular localizations, including different organelles, granules, or membrane fractions. For example, Rab6/Rabkinesin actively transport material along microtubules from the Golgi network to the endoplasmic reticulum (see Science 1998 v279: 580-585; and J Cell Biol 1999 v149:743-759). Receptor-mediated endocytosis can be exploited to facilitate transport to different subcellular localizations. For example, peptides for nuclear transport, such as SV40 peptide or M9 (influenza hemagglutinin peptide), may be used to facilitate nuclear transport. Peptides for endosomal release, such as GALA peptide or HA-2 (influenza hemagglutinin peptide), may be used to facilitate endosomal release. This can be further enhanced by adding chloroquine to buffer the pH and enhance endosomal release.

II. Definitions

For convenience, certain terms employed in the specification, examples, and appended claims are collected here.

A “motor protein binding moiety” or “MPBM” refers to a peptide or other molecule that binds specifically to a motor protein, such as a myosin V protein, a kinesin protein, or a dynein light chain protein. In preferred embodiments, the subject MPBM will bind to its target motor protein with an dissociation constant (K_{d}) of 10⁻⁵ or less, and more preferably 10⁻⁶M, 10⁻⁷M, 10⁻⁸M, 10⁻⁹M, 10⁻¹⁰M, 10⁻¹¹M or less, or most preferably 10⁻¹² or less. The MPBM may also contain several motor protein-binding sequences (MPBSs).

Specifically, a “dynein binding moiety” or “DBM” refers to a peptide or other molecule that binds specifically to a dynein protein, such as a dynein light chain protein. In preferred embodiments, the subject dynein peptide will bind to a dynein protein with an dissociation constant (K_{d}) of 10⁻⁵ or less, and more preferably 10⁻⁶M, 10⁻⁷M, 10⁻⁸M, 10⁻⁹M, 10⁻¹⁰M, 10⁻¹¹M or less, or most preferably 10⁻¹² or less. The DBM may also contain several dynein binding sequences (DBSs).

The term “Motor Protein Therapeutic” as used herein is intended to generically encompass, unless otherwise obvious from its context, the chimeric molecules described herein as including a motor protein binding moiety (comprising at least one motor protein binding sequence) and drug moiety, and includes peptides, peptidomimetics and other small molecule mimics thereof, as well as expressions constructs of such peptides and polypeptides.

The term “Dynein Therapeutic” as used herein is intended to generically encompass, unless otherwise obvious from its context, the chimeric molecules described herein as including a dynein binding moiety (comprising at least one dynein binding sequence) and drug moiety, and includes peptides, peptidomimetics and other small molecule mimics thereof, as well as expressions constructs of such peptides and polypeptides.

The term “linked” means any possible linkage between the motor protein binding moiety and another molecule to be introduced into an organelle or a specific subcellular localization of a eukaryotic cell, e.g., by covalent bonds, hydrogen bonds, ionic interactions, or interaction via a third molecule.

The term “transport into the nucleus” means that the molecule is translocated into the nucleus. Nuclear translocation can be detected by direct and indirect means: Direct observation by fluorescence or confocal laser scanning microscopy is possible when either or both the dynein binding moiety or the translocated molecule are labeled with a fluorescent dye (labeling kits are commercially available, e.g. from Pierce or Molecular Probes). Translocation can also be assessed by electron microscopy if either or both the translocation inducing agent (the nuclear localization peptide) or the translocated molecule are labeled with an electron-dense material such as colloidal gold (Oliver, (1999) Methods Mol. Biol. 115:341-345). Translocation can be assessed in indirect ways if the transported molecule exerts a function in the nucleus. This function can be, but is not limited to, altering the expression of a gene, including the consequences of such gene expression that may be exerted on other cellular molecules or processes, as well as changes in chromatin structure.

Similarly, the term “transport into the organelle” means that the molecule is translocated into the organelle. Organelle translocation can be detected by direct and indirect means: Direct observation by fluorescence or confocal laser scanning microscopy is possible when either or both the motor protein binding moiety or the translocated molecule are labeled with a fluorescent dye (labeling kits are commercially available, e.g. from Pierce or Molecular Probes). Translocation can also be assessed by electron microscopy if either or both the translocation inducing agent (e.g., for the nucleus, the nuclear localization peptide) or the translocated molecule are labeled with an electron-dense material such as colloidal gold (Oliver, (1999) Methods Mol. Biol. 115:341-345). Translocation can be assessed in indirect ways if the transported molecule exerts a function in the organelle.

As used herein, the term “gene” or “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides.

The terms “protein”, “polypeptide” and “peptide” are used interchangeably herein when referring to a gene product, e.g., as may be encoded by a coding sequence.

As used herein, the term “transfection” means the introduction of a nucleic acid, e.g., an expression vector, into a recipient cell by nucleic acid-mediated gene transfer.

“Transcriptional regulatory sequence” is a generic term used throughout the specification to refer to DNA sequences, such as initiation signals, enhancers, and promoters, which induce or control transcription of protein coding sequences with which they are operably linked.

“Operably linked” is intended to mean that the nucleotide sequence is linked to a regulatory sequence in a manner which allows expression of the nucleotide sequence. Regulatory sequences are art-recognized and are selected to direct expression of the subject peptide. Accordingly, the term “transcriptional regulatory sequence” includes promoters, enhancers and other expression control elements. Such regulatory sequences are described in Goeddel; Gene Expression Technology: Methods in Enzymology 185, Academic Press, San Diego, Calif. (1990).

The term “gene construct” refers to a vector, plasmid, viral genome or the like which includes a coding sequence, can transfect cells, preferably mammalian cells, and can cause expression of a polypeptide form of a Dynein Therapeutic.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The terms “chimeric”, “fusion” and “composite” are used to denote a protein, peptide domain or nucleotide sequence or molecule containing at least two component portions which are mutually heterologous in the sense that they are not, otherwise, found directly (covalently) linked in nature. More specifically, the component portions are not found in the same continuous polypeptide or gene in nature, at least not in the same order or orientation or with the same spacing present in the chimeric protein or composite domain. Such materials contain components derived from at least two different proteins or genes or from at least two non-adjacent portions of the same protein or gene. Composite proteins, and DNA sequences which encode them, are recombinant in the sense that they contain at least two constituent portions which are not otherwise found directly linked (covalently) together in nature.

The “growth state” of a cell refers to the rate of proliferation of the cell and/or the state of differentiation of the cell. An “altered growth state” is a growth state characterized by an abnormal rate of proliferation, e.g., a cell exhibiting an increased or decreased rate of proliferation relative to a normal cell.

A “patient” or “subject” to be treated by the subject method can mean either a human or non-human animal.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers. D- and L-α-Amino acids are represented by the following Fischer projections and wedge-and-dash drawings. In the majority of cases, D- and L-amino acids have R- and S-absolute configurations, respectively.

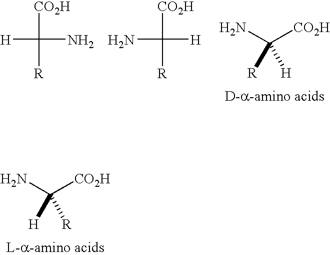

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g. the ability to bind to a motor protein), wherein one or more simple variations of substituents are made which do not adversely affect the efficacy of the compound in binding to a motor protein. In general, the compounds of the present invention may be prepared by the methods illustrated in the general reaction schemes as, for example, described below, or by modifications thereof, using readily available starting materials, reagents and conventional synthesis procedures. Thus, the contemplated equivalents include peptidomimetic or non-peptide small molecule binders of the motor protein. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

As used herein, the term “pharmaceutically acceptable” refers to a carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitoneal, with intravenous being preferred.

III. Description of Certain Preferred Embodiments

A. Motor Protein Binding Moieties

Several motor protein binding motifs have been elucidated.

For example, the dynein light chain-binding motif for target binding to dynein light chain has recently been determined. See Lo et al. J Biol Chem 276:14059-14066, 2001. The conserved amino acid consensus sequence, (K/R)XTQT (SEQ ID NO: 1, “DBP,” dynein-binding peptide), which is present in a number of DLC8 target proteins, can therefore be used to overcome passive diffusion by actively transporting therapeutics toward the nucleus via the dynein motor pathway. DBP can be synthesized with a terminal cysteine residue for conjugation to a nucleic acid carrier (such as polylysine or polyethylenimine) through a crosslinker or directly to DNA via a maleimide labeled, peptide-nucleic acid (PNA) clamp.

Lo et al. also outlines a general scheme for identifying DBMs and MPBMs for use in the instant invention. There are a large number of protein targets known to bind to various subunits, especially the cargo-carrying subunits, of different kinds of motor proteins. Since the protein sequences of these target proteins and their respective motor protein subunits are well-known, it is routine to identify the peptide domains, regions, or motifs on these target proteins responsible for motor protein-binding.

For example, using a series of deletion mutations of a target protein, in vitro binding assay with an appropriate dynein subunit protein (such as IC or DLC8 of cytoplasmic dynein) can quickly identify the DBM on that target protein, just as Lo et al. have demonstrated. Once a specific DBM has been identified, it can be tested for its ability to bind other motor proteins, so that only DBMs specific for a particular type of motor proteins may be selected, depending on use, in one embodiment of the instant invention.

More specifically, the DLC8-binding region was mapped to a highly conserved 10-residue fragment (amino acid sequence SYSKETQTPL, SEQ ID NO: 2) C-terminal to the second alternative splicing site of dynein intermediate chain (IC). Yeast two-hybrid screening using DLC8 as bait identified numerous additional DLC8-binding proteins. Biochemical and mutational analysis of selected DLC8-binding proteins revealed that DLC8 binds to a consensus sequence containing a (K/R)XTQT (SEQ ID NO: 1) motif. The (K/R)XTQT (SEQ ID NO: 1) motif interacts with the common target-accepting grooves of DLC8 dimer. The role of each conserved amino acid residue in this pentapeptide motif in supporting complex formation with DLC8 was systematically studied using site-directed mutagenesis in Lo et al., J. Biol. Chem. 276: 14059-14066, 2001 (incorporated herein by reference).

In certain preferred embodiments, the dynein-binding moiety is a peptide or peptidomimetic. For instance, the dynein-binding moiety can be a peptide (or corresponding peptidomimetic) which includes an amino acid sequence represented in the consensus sequence

| | |
|---|---|
| Xaa1-Xaa2-Thr-Gln-Thr | (SEQ ID NO: 3) |

wherein
Xaa1 represents an amino acid residue with a positively charged sidechain, such as Lys, His or Arg; and
Xaa2 represents an amino acid residue with a polar sidechain, such as Arg, Asn, Asp, Cys, Glu, Gin, His, Lys, Ser or Thr, or a neutral sidechain, such as Ala, Asn, Cys, Gin, Gly, His, Ile, Leu, Met, Phe, Pro, Ser, Thr, Trp, Tyr or Val.

In certain preferred embodiments, Xaa2 represents an amino acid residue with a small neutral sidechain, such as Gly, Ala or Ser. In other preferred embodiments, Xaa2 represents an amino acid residue with a negative polar sidechain, such as Asp or Glu. In certain embodiments, Xaa2 is selected from Ala, Gly, Glu or Ser.

In certain preferred embodiments, the dynein-binding moiety is a peptide, or peptidomimetic thereof, including a sequence selected from

| | |
|---|---|
| KETQT, | (SEQ ID NO: 4) |
| KSTQT, | (SEQ ID NO: 5) |
| KGTQT, | (SEQ ID NO: 6) |
| RSTQT, | (SEQ ID NO: 7) or |
| KATQT. | (SEQ ID NO: 8) |

In certain other embodiments, the dynein-binding moiety (A) is a peptide sequence which binds to DLC8 and is from neuronal nitric-oxide synthase (Jaffrey and Snyder, Science 274: 774-777, 1996; Fan et al., J. Biol. Chem. 273: 33472-33481, 1998), proapoptotic Bcl-2 family protein Bim (Puthalakath et al., Mol. Cell 3: 287-296, 1999), Drosophilia mRNA localization protein Swallow (Schnorrer et al., Nat. Cell Biol. 2: 185-190, 2000), transcriptional regulator IκB (Crepieux et al., Mol. Cell. Biol. 17: 7375-7385, 1997), or postsynaptic scaffold protein GKAP (Naisbitt et al., J. Neurosci. 20: 4524-4534, 2000). In other embodiments the dynein-binding moiety includes a DLC8-binding domain of nNOS, such as a 17-residue peptide fragment from Met-228 to His-244 of nNOS, MKDTGIQVDRDLDGKSH (SEQ ID NO: 9). Fan et al supra; Liang et al. (1999) Nat. Struct. Biol. 6: 735-740. DLC8 is capable of binding to short peptide fragments of ˜10 amino acid residues from its targets. The target peptides bind to DLC8 in an antiparallel β-strand structure by pairing with the β-strand located at the base of each target-accepting groove (Liang et al., Nat. Struct. Biol. 6: 735-740, 1999; Fan et al., J. Mol. Biol. 306: 97-108, 2000). For example, the nine-peptide MSCDKSTQT (SEQ ID NO: 16) from the Bim protein can bind DLC8 (Lo et al., supra).

The dynein-binding moiety can also be a small organic molecule which selectively binds to DLC8. Such small molecules may be obtained by screenings designed for isolating compounds that selectively bind a given subunit of dynein, such as HC, IC, LC8, Tctexl DLC, or roadblock/LC7.

Preferably, the DBM (peptide, small organic molecule or large synthetic molecule) binds to the IC or LC8 of a cytoplasmic dynein.

Preferably, the DBM binds to dynein protein, such as DCL8, with a dissociation constant, K_{d}, of 10⁻⁴M or less, and even more preferably has a K_{d} for binding DCL8 less than 10⁻⁵M, 10⁻⁶M, 10⁻⁷M or even 10⁻⁸M. The measurement of K_{d} between two molecules is well-known in the art of biochemistry. For example, Scatchard plot analysis may be used to measure binding constant. Briefly, dynein proteins may be isolated and conjugated to magnetic beads. The beads (with dynein or other motor proteins) are then exposed to different concentrations of DBP (or MPBM) to allow the binding to occur. Dynein proteins can then be isolated by application of magnet, and the concentration of DBP left in supernatant can then be determined. These data can be used to plot a Scatchard plot for calculating binding constant K_{d}, using, for example, the following formula:

$$\frac{[ {L}_{bound} ]}{[ {L}_{free} ]} = \frac{- [ {L}_{bound} ]}{{K}_{d}} + \frac{[ {R}_{total} ]}{{K}_{d}}$$

Alternatively, Isothermal titration calorimetry may be used to calculate K_{d}. The advantage of this method is that: a) all measurements can be done in solution; b) protein and DBP can be left in “native” form; no tags are required; c) complete thermodynamic information (such as ΔG, ΔH, ΔS and Kₐ) can be obtained.

The K_{d} may be altered for any specific selected MPBM and motor protein. For example, in the case of dynein and DBP (K/R)XTQT (SEQ ID NO: 1), the amino acid X can be optimized by testing all 20 amino acids. The length and type of the spacer may be altered to affect K_{d}. The binding specificity may be further verified by using certain inhibitors of motor protein—MPBM binding. For example, vanadate or Dynein-specific antibody may be used to verify specific Dynein—DBP binding. Alternatively, inhibitors of dynein-mediated transport, such as erythro-9-[3-(2-hydroxy-nonyl)]adenine (dynein inhibitor) or vinblastine or colchicine (microtubule disassembly) may be used to verify that the transport is indeed mediated by motor protein Dynein.

Similarly, other MPBMs can be identified using similar techniques as outlined above. For example, kinesin is a microtubule-activated adenosine triphosphatase (ATPase) of 380 kD (Brady et al., Science 216: 1129, 1982; Vale et al., Cell 42: 39, 1985; Brady, Nature 317: 73, 1985; Schnapp et al. Cell 40: 455, 1985). The kinesin molecule consists of two 120-kD kinesin heavy chains (KHCs) and two 64-kD kinesin light chains (KLCs) (Brady et al., Supra). It has a rod-like structure composed of two globular heads (10 nm in diameter), a stalk, and a fan-like end, with a total length of 80 nm. The globular heads are composed of KHCs that bind to microtubules (Hirokawa, et al., Cell 56: 867, 1989; Scholey et al. Nature 338: 355, 1989); the KLCs constitute the fan-like end (Hirokawa, supra). Complementary DNA (cDNA) encoding Drosophila KHC yields a protein of 975 amino acids in which the NH₂-terminal 350 amino acids form the motor domain (which binds to microtubules), an helical coiled coil-rich stalk domain involved in dimer formation, and a tail domain (Yang et al., Cell 56: 879, 1989). Localization and functional assays indicate that kinesin acts as a plus end-directed microtubule motor involved in anterograde membrane transport (Pfister et al., J. Cell Biol. 108: 1453, 1989; Hollenbeck, ibid., p. 2335; Hirokawa, et al., J. Cell Biol. 114: 295, 1991; Schnapp et al., ibid. 119, 389, 1992; Brady et al., Proc. Natl. Acad. Sci. U.S.A. 87: 1061, 1990).

The kinesin superfamily of proteins plays a major role in this complex organelle transport. A systematic molecular biological search of kinesin superfamily genes coding for proteins containing adenosine triphosphate (ATP)-binding and microtubule-binding consensus sequences led to the discovery of new kinesin superfamily proteins related to organelle transport (KIFs), 11 from mouse brain (Hirokawa, Trends Cell Biol. 6, 135 (1996); Aizawa, et al., J. Cell Biol. 119: 1287, 1992; Hirokawa, Curr. Opin. Neurobiol. 3, 724, 1993) and three from Drosophila (Endow and Hatsumi, Proc. Natl. Acad. Sci. U.S.A. 88, 4424, 1991; Stewart et al., ibid., p. 8470). Motor proteins from Caenorhabditis elegans were identified in mutants with slow and uncoordinated movement [for example, Unc104 (Hall and Hedgecock, Cell 65, 837, 1991; Otsuka, et al., Neuron 6, 113, 1991)] or chemotaxis [Osm3 (Tabish et al., J. Mol. Biol. 247: 377, 1995)]. Further motor proteins (KRP_{85/95}) have been identified in biochemical extracts from sea urchin (Cole, et al., J. Cell Sci. 101, 291, 1992; Cole, et al., Nature 366, 268, 1993). Systematic molecular biological searches have also identified at least two or three members of the dynein superfamily proteins related to the transport of organelles in sea urchin (Gibbons et al., Mol. Biol. Cell 5, 57, 1994), rat (Tanaka et al., J. Cell Sci. 168, 1883, 1995), and human (Vaisberg et al., J. Cell Biol. 133, 831, 1996).

Three major types of kinesin superfamily proteins have been identified according to the position of the motor domain: NH₂-terminal motor domain type, middle motor domain type, and COOH-terminal motor domain type (referred to as N-type, M-type, and C-type, respectively). Of the proteins that have been identified, the KHC, Unc104/KIF1, KIF3/KRP_{85/95}, KIF4, and KIp67A families (N-type), the KIF2 family (M-type), and the KIFC2/C3 family (C-type) are involved in organelle transport.

Conventional kinesins. Conventional KHC itself forms a family. Although three members of this family have been identified in mouse (KIF5A, KIF5B, and KIF5C) (Hirokawa, Trends Cell Biol. 6, 135, 1996; Aizawa, supra; Nakagawa, et al., Proc. Natl. Acad. Sci. U.S.A. 94, 9654, 1997) and two in humans (HsuKHC and HsnKHC) (Navone, et al., J. Cell Biol. 117, 1263, 1992; Niclas et al., Neuron 12, 1059, 1994), only one member has been identified in other metazoans such as sea urchin, Drosophila, and C. elegans (Saxton et al., Cell 64, 1093, 1991; Gho et al., Science 258, 313, 1992).

Because KLCs are localized at the fan-like end of kinesin where it binds to membranous organelles, it is likely that KLCs modulate the binding of cargoes to microtubules (Hirokawa, et al., Cell 56, 867, 1989). KLC cDNAs from several organisms were cloned and sequenced (Cyr et al., Proc. Natl. Acad. Sci. U.S.A. 88, 10114, 1991; Gauger and Goldstein, J. Biol. Chem. 268, 13657, 1993; Wedaman et al., J. Mol. Biol. 231, 155, 1993). The overall structure of KLC has been conserved among various species, and a long series of NH₂-terminal heptad repeats and several imperfect tandem repeats closer to their COOH-termini were identified in KLC.

Metazoan conventional kinesins have been reported to transport numerous membrane cargoes including mitochondria, lysosomes, endoplasmic reticulum, and a subset of anterograde-moving vesicles in axons (Hirokawa, Science 279, 519-526, 1998). Metazoan conventional kinesin also transports nonmembranous cargo, such as mRNAs (Brendza et al., Science 289, 2120-2122, 2000) and intermediate filaments (Prahlad et al., J. Cell Biol. 143, 159-170, 1998). This expanded repertoire of cargoes was made possible by several evolutionary modifications of conventional kinesins. First, metazoans introduced an accessory subunit (kinesin light chains, KLC) that binds to the KHC tail domain. Recent studies revealed that the light and heavy chains mediate distinct cargo interactions. The light chain's tetratricopeptide (TPR) motif region interacts with MAP kinase scaffolding proteins called JIPs (Jun-N-terminal kinase (JNK)-interacting proteins) (Bowman et al., Cell 103, 583-594, 2000; Byrd et al., Neuron 32, 787-800, 2001; Verhey et al., J. Cell Biol. 152, 959-970, 2001), the amyloid precursor protein (APP) on axonally transported membrane vesicles (Kamal et al., Neuron 28, 449-459, 2000), and vaccina virus (Rietdorf et al., Nat. Cell Biol. 3, 992-1000, 2001). In contrast, the tail domain of the heavy chain interacts with the glutamate-receptor-interacting protein (GRIP1) (Setou et al., Nature 417, 83-87, 2002) and the neurofibromatosis protein (Hakimi et al., J. Biol. Chem. 277, 36909-36912, 2002).

Kinesin II was first identified biochemically in sea urchin eggs and found to contain two distinct motor-containing polypeptide chains that come together to form a heterodimer (Cole et al., Nature 366, 268-270, 1993). Heterodimerization is mediated by complementary charge interactions in an extended region of the coiled-coil stalk. Bound to this motor's tail domain is a tightly associated subunit (called KAP) with an armadillo repeat domain that is known to mediate protein-protein interactions. Because of its three distinct subunits, this motor is referred to as heterotrimeric kinesin II. Metazoans also have another kinesin II gene (Osm3/KIF17), which current evidence indicates encodes a protein that forms homodimers (Signor et al., Mol. Biol. Cell 10, 345-360, 1999; Setou et al., Science 288, 1796-1802, 2000) and does not have an associated subunit (referred to here as homodimer kinesin 11).

Heterotrimeric kinesin II is found in two flagellated single cell eukaryotes, Giardia and Chlamydomonas. “Intraflagellar transport” (IFT), the delivery of building blocks (e.g., tubulin, flagellar dyneins, radial spoke proteins) from the base to the tip of the flagella, occurs by the movement of large protein particles along the axonemal microtubules just beneath the plasma membrane (Rosenbaum and Witman, Nat. Rev. Mol. Cell Biol. 3, 813-825, 2002). Metazoans also use heterotrimeric kinesin 11 to power IFT. Mouse knockouts of heterotrimeric kinesin II genes result in ciliary defects, and analyses of these mutant mice have uncovered new functions for cilia in mammals. One consequence of kinesin II knockouts is a developmental defect called situs inversus, a condition in which the heart is frequently on the wrong side of the midline (Nonaka et al., Cell 95, 829-837, 1998; Marszalek et al., Proc. Natl. Acad. Sci. USA 96, 5043-5048, 1999). This phenotype was traced to a failure to form cilia on the embryonic nodal cells; beating of these cilia in normal embryos was subsequently observed and hypothesized to establish a flow and a consequent gradient of a yet undiscovered morphogen involved in left-right axis formation (Nonaka et al., 1998).

Like conventional kinesin, the tail domains of the two motor subunits and nonmotor KAP subunit may participate in cargo interactions. For example, the KAP subunit has been reported to participate in potential cargo interactions with fodrin, a nonmuscle spectrin (Takeda et al., J. Cell Biol. 148, 1255-1265, 2000), the dynactin complex in melanophores (Deacon et al., J. Cell Biol. 160, 297-301, 2003), and the APC protein (Jimbo et al., Nat. Cell Biol. 4, 323-327, 2002). Although only one KAP gene has been described, which can generate two alternatively spliced isoforms, APC was found to bind to one of the isoforms (Jimbo et al., Nat. Cell Biol. 4, 323-327, 2002). Vertebrate neurons also have a third motor subunit (KIF3C) that forms heterodimers only with KIF3A.

Homodimeric kinesin II is only found in metazoans, in contrast to heterotrimeric kinesin II. The mouse homodimeric kinesin II (KIF17) transports NMDA receptor-containing vesicles in dendrites of CNS neurons (Setou et al., Science 288, 1796-1802, 2000), and overexpression of this motor enhances learning and memory in transgenic mice (Wong et al., Proc. Natl. Acad. Sci. USA 99, 14500-14505, 2002). A testes-specific isoform of KIF17 (with relatively few amino acid differences in the tail domain) was reported to bind to and control the intracellular localization of a LIM-only protein called ACT, which is involved in spermatogenesis (Macho et al., Science 298, 2388-2390, 2002). Thus these kinesin-binding proteins may be used to identify conventional kinesin- or kinesin II-specific MPBMs for use in the instant invention.

Unc104/KIF1 The Unc104 motor was discovered in a mutant screen in C. elegans, where null mutations cause paralysis due to a failure to transport synaptic vesicles to the presynaptic terminals of motor neurons (Hall and Hedgecock, Cell 65, 837-847, 1991). The Unc104/KIF1 kinesins have two diagnostic class-conserved features: a conserved insertion in loop 3 near the nucleotide binding pocket, and the presence of a fork head homology (FHA) domain (documented in other proteins to binds phosphothreonine) C-terminal to the motor domain. An unusual property of the Unc104/KIF1 kinesins is that they are predominantly monomeric (Okada et al., Cell 81, 769-780, 1995), in contrast to other kinesins, which are dimeric or tetrameric. However, when concentrated in solution or on membranes, Unc104/KIF1 can dimerize via coiled-coil regions adjacent to the motor domain, and dimerization allows the motor to move processively along microtubules like conventional kinesin (Tomishige et al., Science 297, 2263-2267, 2002). The monomer-to-dimer transition may serve to activate Unc104/KIF1A transport in vivo, and the FHA domain, by virtue of its position in between two coiled-coil domains, could be involved in such a regulatory mechanism.

In lower eukaryotes, Unc104/KIF1 kinesins have been best studied in Ustilago (Wedlich-Soldner et al., EMBO J. 21, 2946-2957, 2002) and Dictyostelium (Pollock et al., J. Cell Biol. 147, 493-506, 1999). In both organisms, gene knockouts of this motor inhibit membrane transport. The tail domains of the Ustilago and Dictyostelium Unc104/KIF1 motors both contain a pleckstrin homology (PH) domain that binds to phosphoinositol lipids and facilitates membrane attachment (Klopfenstein et al., Cell 109, 347-358, 2002). Interestingly, Giardia contains three Unc104/KIF1 type motors, although their roles are not known.

The cargo transporting roles of Unc104/KIF1-type motors also expanded considerably in metazoans, primarily through gene duplication. Thus far, other subunits have not been found complexed with the Unc104/KIF1 motor polypeptide. C. elegans Unc104, Drosophila Klp53D, and mouse KIF1A, by virtue of their C-terminal PH domains, appear to be the closest relatives of the Dictyostelium and Ustilago motors. Interestingly, while the lower eukaryotic Unc104/KIF1A motors have more general roles in membrane trafficking, the metazoan orthologs have taken on the specialized function of transporting synaptic vesicle precursors in the nervous system (Hall and Hedgecock, supra). One of the new metazoan Unc104/KIF1A-type motors (Drosophila kinesin-73, C. elegans CeKLP-4, mouse KIF13B, and human GAKIN) contains a C-terminal cap-gly domain that is known in other proteins to bind tubulin. An intriguing attribute of GAKIN is its binding to the disc large tumor suppressor (Dlg) protein, a membrane-associated guanylate kinase (MAGUK) (Hanada et al., J. Biol. Chem. 275, 28774-28784, 2000).

Further diversity of Unc104/KIF1-type motors in metazoans is achieved through alternative splicing. This has been best documented for the KIF1B gene, where alternative splicing gives rise to motor isoforms with completely different tail domains (Gong et al., Gene 239, 117-127, 1999; Zhao et al., Cell 105, 587-597, 2001). The tail domain of KIF1Bα targets the motor to mitochondria (Nangaku et al., Cell 79, 1209-1220, 1994), while the KIF1Bβ tail targets to synaptic vesicle precursors (Zhao et al., Cell 105, 587-597, 2001). Thus these motors may be used for specific transport of drug therapeutics to these specific organelles or subcellular localizations.

Myosin V The class V myosins were first identified biochemically in vertebrate brain as a myosin-like, calmodulin binding protein and later shown to have motor activity (Cheney et al., Cell 75, 13-23, 1993). The principal structural/sequence feature that characterizes myosin Vs is a long lever arm helix that is stabilized by binding one essential light chain and five calmodulins (Reck-Peterson et al., Biochim. Biophys. Acta 1496, 36-51, 2000). Myosin V's have a conserved <100 residue C-terminal domain (called the dilute, DIL, domain) that is also present in AF6/CNO, a scaffold protein localized at intercellular junctions. Myosin V, at least in vertebrates, binds the same LC8 light chain that is found in cytoplasmic dynein as well as other enzymes such as nitric oxide synthase. This subunit is thought to serve a structural role rather than a cargo binding function under normal circumstances. Nevertheless, this LC8 binding may also be used for myosin V-mediated intracellular transport of drug therapeutics.

In lower eukaryotes, the biological functions of myosin V's have been best studied in S. cerevisiae and S. pombe. In S. cerevisiae, Myo2p delivers various membranes (e.g., secretory vesicles and vacuoles), Kar9 (a protein involved in anchoring microtubles to the bud tip), and Smy1p (a highly divergent kinesin) from the mother to the bud (Reck-Peterson et al., supra). The other S. cerevisiae class V myosin (Myo4p) transports a subset of mRNAs into the bud (Bertrand et al., Mol. Cell 2, 437-445, 1998). In S. pombe, Myo52 (the ortholog of Myo2p in budding yeast) localizes cell wall synthetic enzymes to the tips for polarized growth and orients the mitotic spindle (Win et al., Cell Motil. Cytoskeleton 51, 53-56, 2002). A myosin V-like gene is also present in Malaria, but has not been studied.

In metazoans, myosin Vs also are widely used for organelle transport. Documented examples include endoplasmic reticulum movement in squid axoplasm (Tabb et al., J. Cell Sci. 111, 3221-3234, 1998), melanosome transport in Xenopus melanophores (Rogers and Gelfand, Curr. Biol. 8, 161-164, 1998), and the rapid movement of membranes in plants (the related class XI myosin; Morimatsu et al., Biochem. Biophys. Res. Commun. 270, 147-152, 2000). The most detailed understanding of a metazoan myosin V has been obtained in mice, owing to mutations in this gene, which causes pigmentary dilution due to impaired transport of melanosome granules in melanocytes (Reck-Peterson et al., Biochim. Biophys. Acta 1496, 36-51, 2000). These “dilute” mice also have nervous system defects that may arise from improper localization of smooth endoplasmic reticulum in dendrites.

Three myosin Vs, which exhibit distinct tissue distributions, appear to contribute to the diversity of cargo transport activities of this motor class in vertebrates. A compelling case for alternative splicing contributing to cargo recognition also has been made for myosin Va (Wu et al., Nat. Cell Biol. 4, 271-278, 2002). The transport of melanosomes by this motor has been linked to a specific alternatively spliced exon (exon F) in the myosin V tail that interacts with an adaptor protein (melanophilin) that in turn binds GTP-loaded Rab27a on melanosome membranes (reviewed in Langford, Traffic 3, 859-865, 2002). Mutations in melanophilin and Rab27 produce coat color defects like myosin Va, arguing strongly for the proposed mechanism. Exon B in myosin Va, on the other hand, is neuron-specific and may bind axonal cargo, perhaps via another Rab GTPase.

The above section describes many known target proteins of the various motor proteins. The sequences of these target proteins and their respective motor proteins are well-known in the art, and can be routinely obtained by searching public databases such as PubMed, GenBank, EMBL, DDBJ (DNA Database of Japan), SwissProt, PIR, etc. The NCBI website also offers search engines such as the various BLAST programs for identifying homologs of query proteins or nucleic acids. Once the sequence of these proteins or nucleic acids are obtained, routine molecular biology technique can be used to recombinantly produce the motor protein subunits and their respective target proteins (of fragments thereof) to identify the MPBMs within the targets.

To illustrate, the sequences of almost all motor proteins are well-known in the art or readily obtainable. For example, Table 1 of Vale (Cell 112: 467-480, 2003, all recited references in the Table are incorporated herein by reference) described known conventional kinesin, kinesin II, Unc104/KIF1, mitotic kinesin, C-terminal kinesin, and a number of “other” kinesins; cytoplasmic and axonemal dyne ins; myosin V in a variety of organisms, including Giardia, Malaria, S. cerevisiae, Drosophila, C. elegans, Arabidopsis, Ciona, and human. Public database search using a similar scheme, e.g. searching protein and/or nucleic acid sequence database with conserved kinesin (such as the motor domain) or dynein domains (such as the AAA domain), may yield more motor proteins in other organisms. With the sequences of these kinesin or dynein proteins, it requires merely routine technology in the art to express these motor proteins (or fragments thereof) and test their ability to bind a given target protein (or fragment thereof), either in vitro or in vivo.

Alternatively, other motor protein-binding moieties, including peptides, peptidomimetics, non-peptide small molecules, genes and recombinant polypeptides may be generated using combinatorial techniques using techniques which are available in the art for generating combinatorial libraries of small organic/peptide libraries. See, for example, Blondelle et al. (1995) Trends Anal. Chem. 14:83; the Affymax U.S. Pat. Nos. 5,359,115 and 5,362,899; the Ellman U.S. Pat. No. 5,288,514; the Still et al. PCT publication WO 94/08051; Chen et al. (1994) JACS 116:2661; Kerr et al. (1993) JACS 115:252; PCT publications WO92/10092, WO93/09668 and WO91/07087; and the Lerner et al. PCT publication WO93/20242).

To further illustrate, a combinatorial peptide library can be produced by way of a degenerate library of genes encoding a library of polypeptides which each include at least a portion of potential motor protein binding sequences. For instance, a mixture of synthetic oligonucleotides can be enzymatically ligated into gene sequences such that the degenerate set of potential motor protein binding sequences are expressible as individual polypeptides, or alternatively, as a set of larger polypeptide motor protein therapeutics (e.g. for phage display) each containing a potential motor protein binding sequences therein.

There are many ways by which the library of coding sequences for potential motor protein binding sequences can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential motor protein binding sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations. Such techniques will be generally adaptable for rapid screening of the gene libraries generated by the combinatorial mutagenesis of dynein binding sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques.

In an illustrative embodiment of a screening assay, the motor protein binding gene library can be expressed as a polypeptide motor protein therapeutic on the surface of a viral particle. For instance, in the filamentous phage system, foreign peptide sequences can be expressed on the surface of infectious phage, thereby conferring two significant benefits. First, since these phage can be applied to motor protein at very high concentrations, a large number of phage can be screened at one time. Second, since each infectious phage displays the combinatorial gene product on its surface, if a particular phage is recovered from the cancer cells in low yield, the phage can be amplified by another round of infection. The group of almost identical E. coli filamentous phages M13, fd, and fl are most often used in phage display libraries, as either of the phage gIII or gVIII coat proteins can be used to generate polypeptide motor protein therapeutics without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J. 12:725-734; Clackson et al. (1991) Nature 352:624-628; and Barbas et al. (1992) PNAS 89: 4457-4461).

For example, the recombinant phage antibody system (RPAS, Pharmacia Catalog number 27-9400-01) can be easily modified for use in expressing and screening motor protein binding motif combinatorial libraries of the present invention. For instance, the pCANTAB 5 phagemid of the RPAS kit contains the gene which encodes the phage gIII coat protein. The motor protein binding combinatorial gene library can be cloned into the phagemid adjacent to the gIII signal sequence such that it will be expressed as a gIII polypeptide motor protein therapeutic. After ligation, the phagemid is used to transform competent E. coli TG1 cells. Transformed cells are subsequently infected with M13KO7 helper phage to rescue the phagemid and its candidate motor protein binding gene insert. The resulting recombinant phage contain phagemid DNA encoding a specific candidate motor protein binding moiety, and display one or more copies of the corresponding fusion coat protein. The phage-displayed candidate proteins which are capable of, for example, binding to a motor protein, are selected or enriched by affinity purification. For instance, the phage library can be applied to a column including immobilized DLC8, and unbound phage washed away. The bound phage is then isolated, and if the recombinant phage express at least one copy of the wild type gIII coat protein, they will retain their ability to infect E. coli. Thus, successive rounds of reinfection of E. coli, and affinity maturation can greatly enrich for motor protein binding sequences conditions.

B. Motor Protein Binding Motif Peptidomimetics

In other embodiments, the subject motor protein binding moiety is a peptidomimetics of a motor protein binding motif/sequence. Peptidomimetics are compounds based on, or derived from, peptides and proteins. The motor protein binding peptidomimetics of the present invention typically can be obtained by structural modification of a known motor protein binding moiety sequence using unnatural amino acids, conformational restraints, isosteric replacement, and the like. The subject peptidomimetics constitute the continuum of structural space between peptides and non-peptide synthetic structures; motor protein binding peptidomimetics may be useful, therefore, in delineating pharmacophores and in helping to translate peptides into nonpeptide compounds with the activity of the parent motor protein binding moieties.

Moreover, as is apparent from the present disclosure, mimetopes of the subject motor protein-binding moieties can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in Peptides: Chemistry and Biology, G. R. Marshall ed., ESCOM Publisher: Leiden, Netherlands, 1988)

In addition to a variety of sidechain replacements which can be carried out to generate the subject motor protein-binding peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

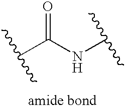

Examples of Surrogates

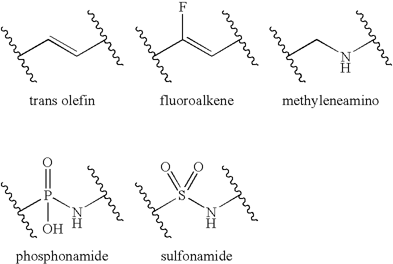

Additionally, peptidomimietics based on more substantial modifications of the backbone of the motor protein-binding sequence can be used. Peptidomimetics which fall in this category include (i) retro-inverso analogs, and (ii) N-alkyl glycine analogs (so-called peptoids).

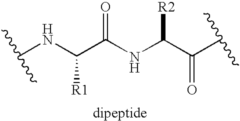

Examples of Analogs

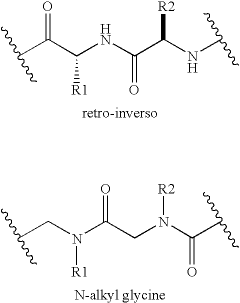

Furthermore, the methods of combinatorial chemistry are being brought to bear on the development of new peptidomimetics. For example, one embodiment of a so-called “peptide morphing” strategy focuses on the random generation of a library of peptide analogs that comprise a wide range of peptide bond substitutes. See, for example, PCT publication WO99/48897 Synthesis Of Compounds And Libraries Of Compounds.

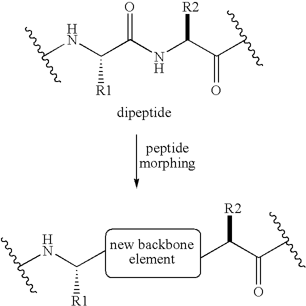

In an exemplary embodiment, the peptidomimetic can be derived as a retro-inverso analog of the peptide

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching The final product, or intermediates thereof, can be purified by HPLC.

In another illustrative embodiment, the peptidomimetic can be derived as a retro-enatio analog of a particular motor protein-binding peptide sequence. Retro-enantio analogs such as this can be synthesized commercially available D-amino acids (or analogs thereof) and standard solid- or solution-phase peptide-synthesis techniques.

In still another illustrative embodiment, trans-olefin derivatives can be made for any of the subject polypeptides. A trans-olefin analog of motor protein-binding moiety can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible couple the pseudodipeptides synthesized by the above method to other pseudodipeptides, to make peptide analogs with several olefinic functionalities in place of amide functionalities. For example, pseudodipeptides corresponding to certain di-peptide sequences could be made and then coupled together by standard techniques to yield an analog of the motor protein-binding moiety which has alternating olefinic bonds between residues.

Still another class of peptidomimetic derivatives include phosphonate derivatives. The synthesis of such phosphonate derivatives can be adapted from known synthesis schemes. See, for example, Loots et al. in Peptides. Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject motor protein-binding peptidomimetics. To illustrate, the motor protein-binding peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject motor protein binding peptidomimetics can be optimized by, e.g., combinatorial synthesis techniques combined with such high throughput screening as described above using affinity maturation of the library by binding to motor protein proteins and selection of specific binding moieties by counterscreening using other cellular proteins.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the motor protein binding domain or inhibiting the interaction between a motor protein binding domain and a motor protein, such as DLC8. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. the predicted mimetope structures can then be produced by, for example, chemical synthesis, recombinant DNA technology, or by isolating a mimetope from a natural source (e.g., plants, animals, bacteria and fungi).

C. Generating Chimeric Entites using Linkers

In certain embodiments, the subject motor protein therapeutics are chimeric polypeptides. In addition to chemical synthesis, techniques for making the subject polypeptide can be adapted from well-known recombinant procedures. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments. Amplification products can subsequently be annealed to generate a chimeric gene sequence (see, for example, Current Protocols in Molecular Biology, Eds. Ausubel et al. John Wiley & Sons: 1992).

The subject motor protein therapeutics can also be generated by synthetic steps, and may include cross-coupling reactions to build up the molecule from the component pieces, e.g., coupling of previously synthesized motor protein-binding moieties and drug moieties. In addition to forming binds directly between functionalities on each of the motor protein-binding moiety and drug moiety, certain of the motor protein therapeutics of the present invention can also be generated using well-known cross-linking reagents and protocols. Merely to illustrate, heterobifunctional cross-linkers can be used to link the various moieties in a stepwise manner. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[β-(4-azidosalicylamido)ethyl]disulfide (BASED) and N-succinimidyl-6(4′-azido-2′-nitrophenyl-amino) hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

D. Non-Covalent Bonding Interaction between MPBMs and Drug Moieties

In certain embodiments of the invention, the motor protein binding moiety A and the drug moiety B are non-covalently associated. Such association can take a variety of forms, including hydrogen bonding, ionic interaction, hydrophobic force, van der Waals interaction. As a result, A and B may be associated with each other by direct binding of A to B; or simultaneous binding of A and B to a third molecule C. The third molecule C may be a protein, a small organic molecule, or a large synthetic molecule. Either A or B or both may contain heterologous sequences (unrelated to their respective MPBM and drug function motifs) that may facilitate A::B interaction.

Merely to illustrate, an MPBM may be covalently linked to a biotin, and a drug moiety may be covalently linked to an avidin or streptavidin (biotin may also be covalently linked to the drug moiety if the drug moiety is at least partly a polypeptide). With a K_{d} of about 10⁻¹⁴M, the biotin-avidin interaction is one of the strongest known biological interactions. The Biotin AviTag™ Technology (AVIDITY, LLC) as described in U.S. Pat. Nos. 5,723,584, 5,874,239 & 5,932,433 can be readily adapted for this purpose. Briefly, Biotin AviTag™ sequence, as described in U.S. Pat. Nos. 5,932,433, 5,874,239 & 5,723,584, is a unique 15-residue peptide that is recognized by biotin ligase (Schatz, Biotechnology 11(10): 1138-1143, October, 1993). In the presence of ATP, the biotin ligase specifically attaches biotin to the lysine residue in this 15-residue sequence known as AviTag™. Using vectors developed by Avidity, LLC, or other equivalent vectors, the Biotin AviTag™ can be genetically fused to a much bigger protein. This feature effectively allows any protein that has been cloned to be tagged with a biotin molecule.

The Biotin AviTag™ system affords several major advantages over the chemical labeling of proteins with biotin:
Because the biotinylation is performed enzymatically, the reaction conditions are very gentle and the labeling is highly specific;
Either in vivo or in vitro biotinylation of proteins is possible;
The Biotin AviTag™ has only one fifth of the bulk of alternative biotinylation tag sequences that are over 85 amino acid residues long (although such alternative biotinylation tag sequences may also be fused to moiety A for the same purpose);
The Biotin AviTag™ can be easily introduced at either the N- or C-terminus or an external loop of any cloned target protein.

Small molecule dimerizers may be used to bring two proteins together. To illustrate, ARIAD Parmaceutical's ARGENT™ homodimerization kit and ARGENT™ heterodimerization kit may be used for this purpose. The ARGENT™ Regulated Homodimerization Kit contains reagents for bringing together two molecules of an engineered fusion protein by adding a small molecule “dimerizer.” The kit can be used to bring together any two proteins that normally do not interact with each other.

There are two classes of dimerizers. Homodimerizers incorporate two identical binding motifs, and can therefore be used to induce association of two proteins containing the same dimerizer-binding motif. Heterodimerizers incorporate two different binding motifs, one on each of the two proteins, and can therefore be used to induce association of the two proteins containing these dimerizer-binding motifs. The ARGENT™ Kits also provides a homodimerizer or a heterodimerizer, and DNA vectors for making appropriate fusion proteins.

The reagents in the ARGENT Kits are based on the human protein FKBP12 (FKBP, for FK506 binding protein) and its small molecule ligands. FKBP is an abundant cytoplasmic protein that serves as the initial intracellular target for the natural product immunosuppressive drugs FK506 and rapamycin. In the original homodimerizer system developed by the Schreiber and Crabtree laboratories (Science 262: 1019-24, 1993), a dimerizer was created by chemically linking two molecules of FK506 in a manner that eliminated immunosuppressive activity. The resulting molecule, called FK1012, was able to crosslink fusion proteins containing wild-type FKBP domains.

A second generation FKBP homodimerizer, AP1510, was subsequently developed by scientists at AR1AD (Amara et al., Proc Natl Acad Sci USA 94: 10618-23, 1997). AP1510 has the advantages of being completely synthetic, as well as being smaller and simpler than FK1012 and more potent in many applications. More recently, ARIAD have improved the affinity and specificity of these molecules further by eliminating their ability to bind to endogenous FKBP. This was achieved by remodeling the FKBP-ligand interface using protein engineering (Clackson et al., Proc Natl Acad Sci USA 95: 10437-42, 1998). The resulting third generation homodimerizers, AP1903 and AP20187, bind with subnanomolar affinity to FKBPs with a single amino acid substitution, Phe36Val (FV), while binding with 1000-fold lower affinity to the wild-type protein. The new system invariably provides more potent activation of homodimerization, and the third-generation ligands have pharmacologic properties suitable for in vivo use. AP20187 and FV form the basis of the reagents provided in the ARGENT Kits.

The AP20187-based system has the advantages of working at lower concentrations, and AP20187 has better pharmacokinetic properties than AP1510, allowing it to be used in vivo.

Other similar systems may also be used to bring two macromolecules together. For example, Lin et al., (J. Am. Chem. Soc., 122, 4247-4248, 2000; also featured in Chem. & Eng. News, 78, 52, 2000) use Dexamethasone-Methotrexate as an efficient chemical inducer of protein dimerization in vivo.

Furthermore, in one embodiment, A-L-B or A::B may take the form of an antibody (e.g., Fab fragment) in which the variable regions of the heavy (V_{H}) and light chain (V_{L}) have been replaced with A and B (either A or B can replace either the V_{H} region or the V_{L} region). For example, soluble proteins comprising an extracellular domain from a membrane-bound protein and an immunoglobulin heavy chain constant region was described by Fanslow et al., J. Immunol. 149:65, 1992 and by Noelle et al., Proc. Natl. Acad. Sci. U.S.A. 89:6550, 1992.

In another embodiment, various oligomerization domains may be employed to bring together the separately synthesized A and B.

One class of such oligomerization domain is leucine zipper. WO 94/10308 A1 and its related U.S. Pat. No. 5,716,805 (all incorporated herein by reference) describes the use of leucine zipper oligomerization domains to dimerize/oligomerize two separate heterologous polypeptides. Each of the two separate heterologous polypeptides is synthesized as a fusion protein with a leucine zipper oligomerization domain. In one embodiment, the leucine zipper domain can be removed from the fusion protein, by cleavage with a specific proteolytic enzyme. In another embodiment, a hetero-oligomeric protein is prepared by utilizing leucine zipper domains that preferentially form hetero-oligomers.

To illustrate, the leucine zipper domain can be removed from the fusion protein, for example by cleavage with a specific proteolytic enzyme. In addition to a leucine zipper sequence and a heterologous protein, such fusion proteins also comprise an amino acid sequence recognized, and cleaved, by a selected proteolytic enzyme. The leucine zipper domain functions to stabilize the recombinant fusion protein during expression and secretion. After purification of the secreted protein, the leucine zipper is enzymatically removed by treating with the proteolytic enzyme. The heterologous protein may then become monomeric. Such a strategy may be used to conditionally inactivate the subject A-L-B or A::B, especially when the originally desired biological activity of the subject A-L-B or A::B is no longer needed after achieving its primary goal.

In addition, one member of A and B may be linked to one of the hetero-oligomerization leucine zipper, while the other member can be linked to the other of the hetero-oligomerization leucine zipper. This would ensure oligomerization of A with B (rather than with itself). U.S. Pat. No. 5,716,805 (incorporated herein by reference) describes in detail about leucine zipper systems for preferentially forming heteroligomers.

Leucine zipper domains were originally identified in several DNA-binding proteins (Landschulz et al., Science 240:1759, 1988). Leucine zipper domain is a term used to refer to a conserved peptide domain present in these (and other) proteins, which is responsible for dimerization of the proteins. The leucine zipper domain (also referred to herein as an oligomerizing, or oligomer-forming, domain) comprises a repetitive heptad repeat, with four or five leucine residues interspersed with other amino acids.

Examples of leucine zipper domains are those found in the yeast transcription factor GCN4 and a heat-stable DNA-binding protein found in rat liver (C/EBP; Landschulz et al., Science 243:1681, 1989). Two nuclear transforming proteins, fos and jun, also exhibit leucine zipper domains, as does the gene product of the murine proto-oncogene, c-myc (Landschulz et al., Science 240:1759, 1988). The products of the nuclear oncogenes fos and jun comprise leucine zipper domains preferentially form a heterodimer (O'Shea et al., Science 245:646, 1989; Turner and Tjian, Science 243:1689, 1989). The leucine zipper domain is necessary for biological activity (DNA binding) in these proteins.

The fusogenic proteins of several different viruses, including paramyxovirus, coronavirus, measles virus and many retroviruses, also possess leucine zipper domains (Buckland and Wild, Nature 338:547,1989; Britton, Nature 353:394, 1991; Delwart and Mosialos, AIDS Research and Human Retroviruses 6:703, 1990). The leucine zipper domains in these fusogenic viral proteins are near the transmembrane region of the proteins; it has been suggested that the leucine zipper domains could contribute to the oligomeric structure of the fusogenic proteins. Oligomerization of fusogenic viral proteins is involved in fusion pore formation (Spruce et al, Proc. Natl. Acad. Sci. U.S.A. 88:3523, 1991). Leucine zipper domains have also been recently report ed to play a role in oligomerization of heat-shock transcription factors (Rabindran et al., Science 259:230, 1993).

Leucine zipper domains fold as short, parallel coiled coils. (O'Shea et al., Science 254:539; 1991) The general architecture of the parallel coiled coil has been well characterized, with a “knobs-into-holes” packing as proposed by Crick in 1953 (Acta Crystallogr. 6:689). The dimer formed by a leucine zipper domain is stabilized by the heptad repeat, designated (abcdeffin according to the notation of McLachlan and Stewart (J. Mol. Biol. 98:293; 1975), in which residues a and d are generally hydrophobic residues, with d being a leucine, which line up on the same face of a helix. Oppositely-charged residues commonly occur at positions g and e. Thus, in a parallel coiled coil formed from two helical leucine zipper domains, the “knobs” formed by the hydrophobic side chains of the first helix are packed into the “holes” formed between the side chains of the second helix.

The leucine residues at position d contribute large hydrophobic stabilization energies, and are important for dimer formation (Krystek et al., Int. J. Peptide Res. 38.2299 1991). Lovejoy et al. recently reported the synthesis of a triple-stranded □-helical bundle in which the helices run up-up-down (Science 259:1288, 1993). Their studies confirmed that hydrophobic stabilization energy provides the main driving force for the formation of coiled coils from helical monomers. These studies also indicate that electrostatic interactions contribute to the stoichiometry and geometry of coiled coils.

Several studies have indicated that conservative amino acids may be substituted for individual leucine residues with minimal decrease in the ability to dimerize; multiple changes, however, usually result in loss of this ability (Landschulz et al., Science 243:1681, 1989; Turner and Tjian, Science 243:1689, 1989; Hu et al., Science 250:1400, 1990). van Heekeren et al. reported that a number of different amino residues can be substituted for the leucine residues in the leucine zipper domain of GCN4, and further found that some GCN4 proteins containing two isoleucine substitutions were weakly active (Nucl. Acids Res. 20:3721, 1992). Mutation of the first and second heptadic leucines of the leucine zipper domain of the measles virus fusion protein (MVF) did not affect syncytium formation (a measure of virally-induced cell fusion); however, mutation of all four leucine residues prevented fusion completely (Buckland et al., J. Gen. Virol. 73:1703, 1992). None of the mutations affected the ability of MVF to form a tetramer.

Recently, amino acid substitutions in the a and d residues of a synthetic peptide representing the GCN4 leucine zipper domain have been found to change the oligomerization properties of the leucine zipper domain (Alber, Sixth Symposium of the Protein Society, San Diego, Calif.). When all residues at position a are changed to isoleucine, the leucine zipper still forms a parallel dimer. When, in addition to this change, all leucine residues at position d are also changed to isoleucine, the resultant peptide spontaneously forms a trimeric parallel coiled coil in solution. Substituting all amino acids at position d with isoleucine and at position a with leucine results in a peptide that tetramerizes. Peptides containing these substitutions are still referred to as leucine zipper domains since the mechanism of oligomer formation is believed to be the same as that for traditional leucine zipper domains such as those described above.

Preparation of fusion proteins are well-known in the art. Fusion proteins are polypeptides that comprise two or more regions derived from different, or heterologous, proteins or peptides. Briefly, fusion proteins can be routinely prepared using conventional techniques of enzyme cutting and ligation of fragments from desired sequences. PCR techniques employing synthetic oligonucleotides may be used to prepare and/or amplify the desired fragments. Overlapping synthetic oligonucleotides representing the desired sequences can also be used to prepare DNA constructs encoding fusion proteins. Fusion proteins can comprise several sequences, including a leader (or signal peptide) sequence, linker sequence, a leucine zipper sequence, or other oligomer-forming sequences, and sequences encoding highly antigenic moieties that provide a means for facile purification or rapid detection of a fusion protein.

Signal peptides facilitate secretion of proteins from cells. An exemplary signal peptide is the amino terminal 25 amino acids of the leader sequence of murine interleukin-7 (IL-7; Namen et al., Nature 333:57]; 1988). Other signal peptides may also be employed furthermore, certain nucleotides in the IL-7 leader sequence can be altered without altering the amino acid sequence. Additionally, amino acid changes that do not affect the ability of the IL-7 sequence to act as a leader sequence can be made. A signal peptide may be added to the fusion A or B, such that when these domains are synthesized by cells from transfected nucleic acids, the secreted A and B will oligomerize to form mature A::B.

A protein of interest may be linked directly to another protein to form a fusion protein; alternatively, the proteins may be separated by a distance sufficient to ensure that the proteins form proper secondary and tertiary structures. Suitable linker sequences (1) will adopt a flexible extended conformation, (2) will not exhibit a propensity for developing an ordered secondary structure which could interact with the functional domains of fusion proteins, and (3) will have minimal hydrophobic or charged character which could promote interaction with the functional protein domains. Typical surface amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, may also be used in the linker sequence. The length of the linker sequence may vary without significantly affecting the biological activity of the fusion protein. Linker sequences are unnecessary where the proteins being fused have non-essential N- or C-terminal amino acid regions which can be used to separate the functional domains and prevent steric interference. Exemplary linker sequences are described in U.S. Pat. Nos. 5,073,627 and 5,108,910, the disclosures of which are incorporated by reference herein. A preferred linker is one or more repeats of the penta-peptide Gly-Gly-Gly-Gly-Ser. In addition, WO 01/53480 discusses optimizing the use of flexible linkers between domains, the entire content of which is incorporated herein by reference.

Many other so-called “bundling domains” exist which perform essentially the same function of the above-described leucine zipper domains to bring together A and B. For example, WO 99/10510 A2 (incorporated herein by reference) describes bundling domains include any domain that induces proteins that contain it to form multimers (“bundles”) through protein-protein interactions with each other or with other proteins containing the bundling domain. Examples of these bundling domains include domains such as the lac repressor tetramerization domain, the p53 tetramerization domain, the leucine zipper domain, and domains derived therefrom which retain observable bundling activity. Proteins containing a bundling domain are capable of complexing with one another to form a bundle of the individual protein molecules. Such bundling is “constitutive” in the sense that it does not require the presence of a cross-linking agent (i.e., a cross-linking agent which doesn't itself contain a pertinacious bundling domain) to link the protein molecules.

As described above, bundling domains interact with like domains via protein-protein interactions to induce formation of protein “bundles.” Various order oligomers (dimers, trimers, tetramers, etc.) of proteins containing a bundling domain can be formed, depending on the choice of bundling domain.

One example of a dimerization domain, as described above, is the leucine zipper (LZ) element. Leucine zippers have been identified, generally, as stretches of about 35 amino acids containing 45 leucine residues separated from each other by six amino acids (Maniatis and Abel (1989) Nature 341:24-25). Exemplary leucine zippers occur in a variety of eukaryotic DNA binding proteins, such as GCN4, C/EI3P, c-Fos, c-Jun, c-Myc and c-Max. Other dimerization domains include helix-loop-helix domains (Murre, C. et al. (1989) Cell 58:537544).

Dimerization domains may also be selected from other proteins, such as the retinoic acid receptor, the thyroid hormone receptor or other nuclear hormone receptors (Kurokawa et al. (1993) Genes Dev. 7:1423-1435) or from the yeast transcription factors GAL4 and HAP1 (Marmonstein et al. (1992) Nature 356:408-414; Zhang et al. (1993) Proc. Natl. Acad. Sci. USA 90:2851-2855). Dimerization domains are further described in U.S. Pat. No. 5,624,818 by Eisenman.

In one embodiment, incorporation of a tetramerization domain within a fusion protein leads to the constitutive assembly of tetrameric clusters or bundles. The E. coli lactose repressor tetramerization domain (amino acids 46-360; Chakerian et al. (1991) J. Biol. Chem. 266.1371; Alberti et al. (1993) EMBO J. 12:3227; and Lewis et al. (1996) Nature 271:1247), illustrates this class. Other illustrative tetramerization domains include those derived from residues 322-355 of p53 (Wang et al. (1994) Mol. Cell. Biol. 14:5182; Clore et al. (1994) Science 265:386) see also U.S. Pat. No. 5,573,925 by Halazonetis.

Other bundling domains can be derived from the Dimerization cofactor of hepatocyte nuclear factor-1 (DCoH). DCoH associates with specific DNA binding proteins and also catalyses the dehydration of the biopterin cofactor of phenylalanine hydroxylase. DCoH is a tetramer. See e.g. Endrizzi, J. A., Cronk, J. D., Wang, W., Crabtree, G. R and Alber, T. (1995) Science 268,556559; Suck and Ficner (1996) FEBS Lett 389(1):3-39; Standmann, Senkel and Ryffel (1998) Int J Dev Biol 42(1):53-59 The bundling domain may comprise a naturally-occurring peptide sequence or a modified or artificial peptide sequence. Sequence modifications in the bundling domain may be used to increase the stability of bundle formation or to help avoid unintended bundling with native protein molecules in the engineered cells which contain a wild-type bundling domain.

For example, sequence substitutions that stabilize oligomerization driven by leucine zippers are known (Krylov et al. (1994) cited above; O'Shea et al. (1992) cited above). To illustrate, residues 174 or 175 of human p53 may be replaced by glutamine or leucine, respectively. To illustrate sequence modifications aimed at avoiding unintended bundling with endogenous protein molecules, the p53 tetramerization domain may be modified to reduce the likelihood of bundling with endogenous p53 proteins that have a wild-type p53 tetramerization domain, such as wild-type p53 or tumor-derived p53 mutants. Such altered p53 tetramerization domains are described in U.S. Pat. No. 5,573,925 by Halazonetis and are characterized by disruption of the native p53 tetramerization domain and insertion of a heterologous bundling domain in a way that preserves tetramerization.

Disruption of the p53 tetramerization domain involving residues 335-348, or a subset of these residues, sufficiently disrupts the function of this domain so that it can no longer drive tetramerization with wild-type p53 or tumor-derived p53 mutants. At the same time, however, introduction of a heterologous dimerization domain reestablishes the ability to form tetramers, which is mediated both by the heterologous dimerization domain and by the residual portion of the p53 tetramerization domain sequence.

Other suitable bundling domains can be readily selected or designed by the practitioner, including semi-artificial bundling domains, such as variants of the GCN4 leucine zipper that form tetramers (Alberti et al. (1993) EMBO J. 12:3227-3236; Harbury et al. (1993) Science 262:1401-1407; Krylov et al. (1994) EMBO J. 13:2849-2861). The tetrameric variant of GCN4 leucine zipper described in Harbury et al. (1993), supra, has isoleucines at positions d of the coiled coil and leucines at positions a, in contrast to the original zipper which has leucines and valines, respectively.

The choice of bundling domain can be based, at least in part, on the desired conformation of the bundles. For instance, the GCN4 leucine zipper drives parallel subunit assembly [Harbury et al. (1993), cited above), while the native p53 tetramerization domain drives antiparallel assembly [Clore et al. (1994) cited above; Sakamoto et al. (1994) Proc. Natl. Acad. Sci. USA 91:8974-8978].

In addition, a variety of techniques are available for identifying other naturally occurring bundling domains, as well as for selecting bundling domains derived from mutant or otherwise artificial sequences. See, for example, Zeng et al. (1997) Gene 185:245; O'Shea et al. (1992) Cell 68:699-708; Krylov et al. [cited above].

In applications of the invention involving the genetic engineering of cells within (or for use within) whole animals, the use of peptide sequence derived from that species is preferred when possible. For instance, for applications involving human gene therapy, use of bundling domains derived from human proteins may minimize the risk of immunogenic reactions. However, in some cases the use of bundling domains of human origin may induce interactions between the fusion proteins and the endogenous protein from which the bundling domain was derived, i.e., leading to unwanted bundling of fusion proteins with the endogenous protein containing the identical bundling domain. Such interactions, in addition to inhibiting target gene expression, may also have other adverse effects in the cell, e.g., by interfering with the function of the endogenous protein from which the bundling domain was derived.

Approaches for avoiding unwanted bundling of fusion proteins of this invention with endogenous proteins include using a bundling domain which is (a) heterologous to the host organism, (b) expressed by the host organism but only (or predominantly) in cells or tissues other than those which will express the fusion proteins, or (c) engineered through modification in peptide sequence such that it bundles preferentially with itself rather than with an endogenous bundling domain (see below).

The first approach is illustrated by the use of a bacterial lac repressor tetramerization domain in human cells.

The second approach requires the use of a bundling domain derived from a protein which is not expressed in the cells or tissues which are to be engineered to express the fusion protein(s) of this invention, at least not at a level which would cause undue interference with the bundling application or with normal cell function. Fusion proteins containing a bundling domain derived from an endogenous protein expressed selectively or preferentially in one tissue could be expressed in a different tissue without any adverse effects. For example, to regulate gene expression in human muscle, fusion proteins containing bundling domains from a protein expressed in liver, brain or some other tissue or tissues-but not in muscle—can be expressed in muscle cells without undue risk of mismatched bundling.

In the third approach, and as noted previously, the binding specificity of the bundling domain is engineered by alterations in peptide sequence to replace (in whole or part) bundling activity for proteins containing the wild-type bundling domain with bundling activity for proteins containing the modified peptide sequence. For example, U.S. Pat. No. 6,495,346 (entire contents incorporated herein by reference) describes oligomerization domains which are mutated to work with one and other but no longer interact with native sequences. Specifically, U.S. Pat. No. 6,495,346 describes a mutated dimerization domain having been derived by mutation of a naturally occurring dimerization domain. It is possible for this mutated dimerization domain to interact specifically with a complementary mutated dimerization domain, which is also derived by mutation of its naturally occurring counterpart. The specification of the patent describes in detail a general method of generating such kind of complementary mutated dimerization domains. Exemplary dimerization domains include mutated leucine zipper dimerization domains of c-fos and c-jun, such as the c-fos E167K/c-jun K283E pair; the c-fos E172K/c-jun K288E pair; c-fos E181K/c-jun K302E pair.

Several examples of tissue-specific bundling domains which could be used in the practice of this invention include bundling domains derived from the Retinoid X receptor, (Kersten, S., Reczek, P. R and N. Noy (1997) J. Biol. Chem. 272, 29759-29768); Dopamine D3 receptor (Nimchinsky, E. A., Hof, P. R., Janssen, W. G. M., Morrison, J. H and C. Schmauss (1997) J. Biol. Chem. 272, 29229-29237); Butyrylcholinesterase (Blong, R. M., Bedows, Eand O. Lockridge (1997) Biochem. J. 327, 747-757); Tyrosine Hydroxylase (Goodwill, K. E., Sabatier, C., Marks, C., Raag, R., Fitzpatrick, P. F and R. C. Stevens (1997) Nat. Struct. Biol 7, 578-585). Bcr (McWhirter, J. R., Galasso, D. L and J. Y. Wang (1993) Mol. Cell. Biol. 13, 7587-7595); and Apolipoprotein E (Westerlund, J. A and K. H. Weisgraber (1993) J. Biol. Chem. 268,15745-15750).

In yet another embodiment, A and B may each be fused to a “ligand binding domain,” which, upon binding to a small molecule, will bring A and B together (“small molecule-mediated oligomerization”).

Fusion proteins containing a ligand binding domain for use in practicing this invention can function through one of a variety of molecular mechanisms.

In certain embodiments, the ligand binding domain permits ligand-mediated crosslinking of the fusion protein molecules bearing appropriate ligand binding domains. In these cases, the ligand is at least divalent and functions as a dimerizing agent by binding to the two fusion proteins and forming a cross-linked heterodimeric complex which activates target gene expression. See e.g. WO 94/18317, WO 96/20951, WO 96/06097, WO 97/31898 and WO 96/41865.

In the cross-linking-based dimerization systems the fusion proteins can contain one or more ligand binding domains (in some cases containing two, three, four, or more of such domains) and can further contain one or more additional domains, heterologous with respect to the ligand binding domain, including e.g. A or B of the subject A::B/A-L-B.

In general, any ligand/ligand binding domain pair may be used in such systems. For example, ligand binding domains may be derived from an immunophilin such as an FKBP, cyclophilin, FRB domain, hormone receptor protein, antibody, etc., so long as a ligand is known or can be identified for the ligand binding domain.

For the most part, the receptor domains will be at least about 50 amino acids, and fewer than about 350 amino acids, usually fewer than 200 amino acids, either as the natural domain or truncated active portion thereof. Preferably the binding domain will be small (<25 kDa, to allow efficient transfection in Viral vectors), monomeric, nonimmunogenic, and should have synthetically accessible, cell permeant, nontoxic ligands as described above.

Preferably the ligand binding domain is for (i.e., binds to) a ligand which is not itself a gene product (i.e., is not a protein), has a molecular weight of less than about 5 kD and preferably less than about 2.5 kD, and optionally is cell permeant. In many cases it will be preferred that the ligand does not have an intrinsic pharmacologic activity or toxicity which interferes with its use as an oligomerization regulator.

The DNA sequence encoding the ligand binding domain can be subjected to mutagenesis for a variety of reasons. The mutagenized ligand binding domain can provide for higher binding affinity, allow for discrimination by a ligand between the mutant and naturally occurring forms of the ligand binding domain, provide opportunities to design ligand-ligand binding domain pairs, or the like. The change in the ligand binding domain can involve directed changes in amino acids known to be involved in ligand binding or with ligand-dependent conformational changes. Alternatively, one may employ random mutagenesis using combinatorial techniques. In either event, the mutant ligand binding domain can be expressed in an appropriate prokaryotic or eukaryotic host and then screened for desired ligand binding or conformational properties. Examples involving FKBP, cyclophilin and FRB domains are disclosed in detail in WO 94/18317, WO 96/06097, WO 97/31898 and WO 96/41865. For instance, one can change Phe36 to Ala and/or Asp37 to Gly or Ala in FKBP12 to accommodate a substituent at positions 9 or 10 of the ligand FK506 or FK520 or analogs, mimics, dimers or other derivatives thereof. In particular, mutant FKBP12 domains which contain Val, Ala, Gly, Met or other small amino acids in place of one or more of Tyr26, Phe36, Asp37, Tyr82 and Phe99 are of particular interest as receptor domains for FK506-type and FK type ligands containing modifications at C9 and/or C10 and their synthetic counterparts (see, e.g., WO97/31898). Illustrative mutations of current interest in FKBP domains also include the following:

| | | | |
|---|---|---|---|
| F36A | Y26V | F46A | W59A |
| F36V | Y26S | F48H | H87W |
| F36M | D37A | F48L | H87R |
| F36S | I90A | F48A | F36V/F99A |
| F99A | I91A | E54A/F36V/F99G | F99G |
| F46H | E54K/F36M/F99A | Y26A | F46L |
| V55A | F36M/F99G | | |

Table 1: Entries identify the native amino acid by single letter code and sequence position, followed by the replacement amino acid in the mutant. Thus, F36V designates a human FKBP12 sequence in which phenylalanine at position 36 is replaced by valine. F36V/F99A indicates a double mutation in which phenylalanine at positions 36 and 99 are replaced by valine and alanine, respectively.

Illustrative examples of domains which bind to the FKBP:rapamycin complex (“FRBs”) are those which include an approximately 89-amino acid sequence containing residues 2025-2113 of human FRAP. Another FRAP-derived sequence of interest comprises a 93 amino acid sequence consisting of amino acids 2024-2113. Similar considerations apply to the generation of mutant FRAP-derived domains which bind preferentially to FKBP complexes with rapamycin analogs (rapalogs) containing modifications (i.e., are ‘bumped’) relative to rapamycin in the FRAP-binding portion of the drug. For example, one may obtain preferential binding using rapalogs bearing substituents; other than —OMe at the C7 position with FRBs based on the human FRAP FRB peptide sequence but bearing amino acid substitutions for one of more of the residues Tyr2038, Phe2039, Thr2098, Gln2099, Trp2101 and Asp2102. Exemplary mutations include Y2038H, Y2038L, Y2038V, Y2038A, F2039H, F2039L, F2039A, F2039V, D2102A, T2098A, T2098N, T2098L, and T2098S. Rapalogs bearing substituents; other than —OH at C28 and/or substituents other than ═O at C30 may be used to obtain preferential binding to FRAP proteins bearing an amino acid substitution for Glu2032. Exemplary mutations include E2032A and E2032S. Proteins comprising an FRB containing one or more amino acid replacements at the foregoing positions, libraries of proteins or peptides randomized at those positions (i.e., containing various substituted amino acids at those residues), libraries randomizing the entire protein domain, or combinations of these sets of mutants are made using the procedures described above to identify mutant FRAPs that bind preferentially to bumped rapalogs.

Other macrolide binding domains useful in the present invention, including mutants thereof, are described in the art. See, for example, WO96/41865, WO96/136131 WO96/0611 11 WO96/061 1 01 WO96/060971 WO96/127961 WO95/053891 WO95/026842.

The ability to employ in vitro mutagenesis or combinatorial modifications of sequences encoding proteins allows for the production of libraries of proteins which can be screened for binding affinity for different ligands. For example, one can randomize a sequence of 1 to 5, 5 to 10, or 10 or more codons, at one or more sites in a DNA sequence encoding a binding protein, make an expression construct and introduce the expression construct into a unicellular microorganism, and develop a library of modified sequences. One can then screen the library for binding affinity of the encoded polypeptides to one or more ligands. The best affinity sequences which are compatible with the cells into which they would be introduced can then be used as the ligand binding domain for a given ligand. The ligand may be evaluated with the desired host cells to determine the level of binding of the ligand to endogenous proteins. A binding profile may be determined for each such ligand which compares ligand binding affinity for the modified ligand binding domain to the affinity for endogenous proteins. Those ligands which have the best binding profile could then be used as the ligand. Phage display techniques, as a non-limiting example, can be used in carrying out the foregoing.

In other embodiments, antibody subunits, e.g. heavy or light chain, particularly fragments, more particularly all or part of the variable region, or single chain antibodies, can be used as the ligand binding domain. Antibodies can be prepared against haptens which are pharmaceutically acceptable and the individual antibody subunits screened for binding affinity. cDNA encoding the antibody subunits can be isolated and modified by deletion of the constant region, portions of the variable region, mutagenesis of the variable region, or the like, to obtain a binding protein domain that has the appropriate affinity for the ligand. In this way, almost any physiologically acceptable hapten can be employed as the ligand. Instead of antibody units, natural receptors can be employed, especially where the binding domain is known. In some embodiments of the invention, a fusion protein comprises more than one ligand binding domain. For example, a DNA binding domain can be linked to 2, 3 or 4 or more ligand binding domains. The presence of multiple ligand binding domains means that ligand-mediated cross-linking can recruit multiple fusion proteins containing transcription activation domains to the DNA binding domain-containing fusion protein.

Ligands of the invention: In various embodiments where a ligand binding domain for the ligand is endogenous to the cells to be engineered, R is often desirable to alter the peptide sequence of the ligand binding domain and to use a ligand which discriminates between the endogenous and engineered ligand binding domains. Such a ligand should bind preferentially to the engineered ligand binding domain relative to a naturally occurring peptide sequence, e.g., from which the modified domain was derived. This approach can avoid untoward intrinsic activities of the ligand. Significant guidance and illustrative examples toward that end are provided in the various references cited herein.

Cross-linking/dimerization systems Any ligand for which a binding protein or ligand binding domain is known or can be identified may be used in combination with such a ligand binding domain in carrying out this invention.

Extensive guidance and examples are provided in WO 94/18317 for ligands and other components useful for cross-linked oligomerization-based systems. Systems based on ligands for an immunophilin such as FKBP, a cyclophilin, and/or FRB domain are of special interest. Illustrative examples of ligand binding domain/ligand pairs that may be used for cross-linking include, but are not limited to: FKBP/FK1012, FKBP/synthetic divalent FKBP ligands (see WO 96/06097 and WO 97/31898), FRB/rapamycin or analogs thereof: FKBP (see e.g., WO 93/33052, WO 96/41865 and Rivera et al, “A humanized system for pharmacologic control of gene expression”, Nature Medicine 2(9):1028-1032 (1997)), cyclophilin/cyclosporin (see e.g. WO 94/18317), FKBP/FKCsA/cyclophilin (see e.g. Belshaw et al, 1996, PNAS 93:4604-4607), DHFR/methotrexate (see e.g. Licitra et al, 1996, Proc. Natl. Acad. Sci. USA 93:12817-12821), and DNA gyrase/coumermycin (see e.g. Farrar et al, 1996, Nature 383:178-181). Numerous variations and modifications to ligands and ligand binding domains, as well as methodologies for designing, selecting and/or characterizing them, which may be adapted to the present invention are disclosed in the cited references.

In certain other embodiments, the third molecule may also be a protein that binds to both A and B.

E. Exemplery Enbodiments of Moiety B

In principal, the instant invention can be used to selectively and actively deliver any kinds of drug moieties, as long as they can be coupled to the selected MPBMs.

Merely to illustrate, B can be nucleic acid, polypeptide, other organic molecules, or large synthetic molecules.

For example, B can be an oligonucleotide, such as an antisense oligonucleotide, with or without modification for enhanced solubility, cellular uptake, membrane translocation, and/or in vivo stability. The nucleic acid can be linked to the MPBM via a peptide-nucleic acid (PNA) clamp. “Peptide Nucleic Acids: Protocols and Applications” (Nielsen Ed., published by Horizon Scientific Press, 32 Hewitts Lane, Wymondham, NR180JA, U.K. ISBN 1-898486-16-6 (hbk)) is a book that contains state-of-the-art protocols and applications on all aspects of Peptide Nucleic Acids. Concepts are explained clearly and in practical terms and each chapter contains concise background information. The book is written by leading experts in the field, and is a complete reference work on this area of research. The book provides a complete overview of the scientific theory, applications of PNA, comprehensive background information, synthesis of PNA, and the numerous uses of PNA in biological science.

Similarly, other nucleic acids, such as siRNA or plasmids encoding various protein and/or nucleic acid products may be coupled in a similar fashion to MPBMs.

Insert Therapeutic's proprietary drug delivery technology, Cyclosert™, may also be used to deliver various drug moieties of any size ranging from small-molecule drugs to plasmid DNA The Cyclosert™ technology platform is based upon cup-shaped cyclic repeating molecules of glucose known as cyclodextrins. The “cup” of the cyclodextrin molecule can form “inclusion complexes” with other molecules, making it possible to combine the Cyclosert™ polymers with other moieties to enhance stability or to add targeting ligands. In addition, cyclodextrins have generally been found to be safe in humans (individual cyclodextrins currently enhance solubility in FDA-approved oral and IV drugs) and can be purchased in pharmaceutical grade on a large scale at low cost.

Modified cyclodextrin molecules has been used as building blocks to develop a broad range of polymers. Beginning with a “core” monomer unit comprised of a difunctionalized cyclodextrin molecule, we link it with one of a variety of other monomers depending on the therapeutic payload, use or desired characteristics of the resulting polymer. These polymers are extremely water soluble, non-toxic and non-immunogenic at therapeutic doses, even when administered repeatedly. The polymers can easily be adapted to carry a wide range of small-molecule therapeutics at drug loadings that can be significantly higher than liposomes. Additionally, Cyclosert™ polymers can be tuned to be neutral, positively charged or negatively charged. This feature is unique to the Cyclosert™ technology and provides great flexibility for formulation and delivery.

In contrast to passive drug carriers that degrade over time, Cyclosert™ polymers respond to biological mechanisms and micro-environmental conditions. Thus, they can be designed to release their drug payload at the appropriate time and in the appropriate place. By fine-tuning interaction between the polymers, or targeting agent, and surface receptors on the targeted cells, it is possible to ensure specific uptake into target cells—an objective that have been verified in animal models.

The applicants have accomplished intracellular delivery of small-molecule drugs, plasmid DNA and oligonucleotides (including siRNA, DNAzymes, ribozymes and chimeric oligonucleotides) with Cyclosert™. The applicants have also successfully modified the Cyclosert™ delivery system to include targeting ligands and confirmed receptor-mediated intracellular uptake of a Cyclosert™-DNA complex into specific target cells (both to tumor cells and to specific organs) in in vivo studies following systemic administration. The Cyclosert™ technology can be combined with motor protein-assisted intracellular transport to more effectively deliver drug moiety B. For example, the cyclodextran cup can be effectively coupled to the —SH group of Cys, which can be the terminal residue of MPBM.

In certain embodiments, B can be an agent that interacts with a transcription factor, a histone, an enzyme specifically localized within an organelle, or other protein or protein complex which interact with DNA and regulate gene expression or chromatin structure. Such targets can include cytoplasmic enzymes, nuclear hormone/steroid receptors (such as receptors for glucocorticoids, mineralocorticoids, sex hormones or ecdysone), histone acetylases or deacetylases, DNA methyltransferases and other enzymes which covalently modify DNA, kinases (such as cyclin dependent kinases), phosphatases (such as cdc25 phosphatases), proteases, lipases, RNA polymerases, DNA polymerases, DNA primases, DNA topoisomerases, DNA helicases, nucleases, ATPases (such as chromatin remodeling ATPases), and the like. The agent can be an inhibitor of an intrinsic enzymatic activity, an inhibitor (or potentiator) of protein-protein, protein-DNA and/or protein-lipid interactions, an intercalating agents (including fluorescent dyes) or the like. In some cases, the agent can be a nucleic acid, such as a decoy sequence which binds to a transcription factor or repressor, an antisense sequence, or a double stranded RNA interference construct. The subject invention also contemplates that the agent can be a molecule which interacts with and alters the structure changes of the nuclear envelope.

In certain other embodiments, moiety B may be drug delivery systems for small molecules. Such delivery system may include microspheres, liposomes, etc. For example, U.S. Pat. No. 5,470,311 describes that microspheres comprising biodegradable polymers, ranging in size from less than 45 μm to more than 250 μm, may be used to deliver drug molecules.

These drug-containing nanoparticles (e.g. microspheres) may be coupled to MPBMs to generate the therapeutics of the instant invention. For example, any given MPBM moiety may be engineered to contain a terminal Cys, which may be used to couple the MPBM to the surface of the microsphere with —NH₂ groups. To prevent steric hindrance, a spacer sequence may be introduced between the microsphere and the MPBM. The spacer may be the frequently used (Gly₃Ser)ₙ spacer (SEQ ID NO: 15), or any other appropriate polypeptides. The surface of the microsphere may be chosen or modified to contain —COO⁻, —NH₃, —OH, PEG of various lengths, or other oligosaccharides.

The property of the microspheres may also be an important consideration. For example, Poly-(L-lysine) (or PLL) has poor endosomal release, while Poly(ethylenimine) (or PEI) is more efficient in terms of endosome release.

The surface of the microspheres may also be modified to contain certain target ligands for specific receptor-mediated endocytosis. For example, microspheres modified by galactose may be used for hepatocytes targeting, while RGD-modification is suitable for cells that have integrin receptor. Similarly, folate-modifed microspheres may be used for cells with folate receptors on the surface.

The size of the microspheres may be controlled or modified to achieve optimal result. For example, FRAP (Fluorescence recovery after photobleaching) or gel electrophoresis may be used to test the mobility of a particular microsphere particle in cytoplasm, which is affected by factors such as particle size, surface charge, hydrophobicity/hydrophilicity, etc. For example, a nanoparticle may be fluorescently labeled for use in Fluorescence Recovery After Photobleaching. For this purpose, amine-modified nanospheres can be purchased from Molecular Probes with particle diameter of 20 nm, 40 nm, 100 nm, 200 nm and 1000 nm. Nanospheres can then be labeled with, for example, Alexa488− NHS. Alexa488 has similar excitation/emission to fluorescein but is pH-insensitive between pH 4-10. The remaining amine groups can be capped with glycidol. The fluorescently-labeled nanoparticles are then microinjected into the cytoplasm of mammalian cells, and FRAP analysis can then be carried out with Scanning Laser Confocal Microscopy (SLCM). For example, Hiraoke et al. used FRAP to study the mobility of mutant GFP-tagged emerin in cytoplasm.

The nanoparticle may be conjugated to a selected MPBM, such as DBM, either directly or via a spacer. For example, if the nanoparticle has multiple —NH₂ groups at the surface, it may be directly conjugated to synthesized (or purified) DBM, which may contain a terminal Cys and/or a spacer (see below).

Peptide Synthesis for DBP:

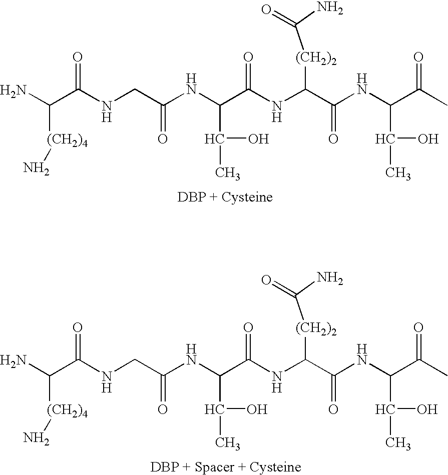

DBP Conjugation to Nanoparticle

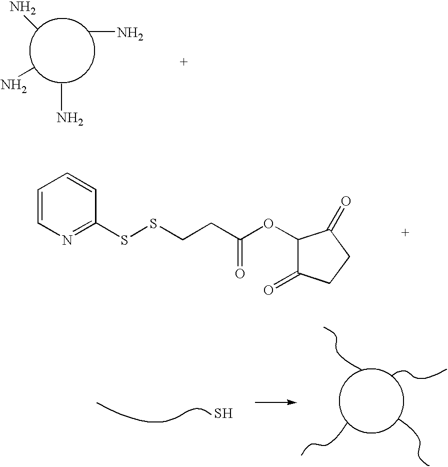

The surface property of the microspheres may also be controlled or modified to achieve optimal result. For example, the surface of a particular nanoparticle may be modified to contain predominantly one type of chemical groups, such as —COO⁻, —NH₂⁺, —OH, -polyethylene glycol of various lengths, or -oligosaccharides. Estimation of the binding between these modified nanoparticles to cytoplasmic extracts can be carried out by turbidity measurements. In addition, nanoparticle modified by a specific chemical group may also be used to determine what kinds of proteins tend to bind to the modified surface. This can be achieved by incubating the modified nanospheres with, for example, cytoplasm extract, isolating nanospheres by ultracentrifugation in a sucrose gradient; displacing proteins with 10% SDS; and running proteins on electrophoresis gel. Various techniques, such as mass spectrometry sequencing of the separated proteins may be used to determine the identity of the proteins bound to the modified nanospheres.

Alternatively, FRAP may also be used to determine the mobility of nanoparticles in cytoplasm.

F. Transcellular and Transmembrane Functionalities

The motor protein therapeutic (either A or B or both motifs) may also include one or more functionalities that promote uptake by target cells, e.g., promote the initial step of uptake from the extracellular environment or promote transmembrane transport, e.g. promote release from endosomal vesicles or transport across organelle membranes. In one embodiment, the motor protein therapeutic includes an “internalizing peptide” which drives the translocation of the motor protein therapeutic across a cell membrane in order to facilitate intracellular localization. The internalizing peptide, by itself, is capable of crossing a cellular membrane by, e.g., transcytosis, at a relatively high rate. The internalizing peptide is conjugated, e.g., as a polypeptide motor protein therapeutic, to a motor protein therapeutic.

In one embodiment, the internalizing peptide is derived from the Drosophila antepennepedia protein, or homologs thereof. The 60 amino acid long homeodomain of the homeo-protein antepennepedia has been demonstrated to translocate through biological membranes and can facilitate the translocation of heterologous peptides and organic compounds to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193. The present invention contemplates a motor protein therapeutic including at least a portion of the antepennepedia protein (or homolog thereof) sufficient to increase the transmembrane transport.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8). Peptides or analogs that include a sequence present in the highly basic region, such as CFITKALGISYGRKKRRQRRRPPQGS (SEQ ID NO: 10), can be used in the motor protein therapeutic to aid in internalization.

Another exemplary motor protein therapeutic can be generated to include a sufficient portion of mastoparan (T. Higashijima et al., (1990) J. Biol. Chem. 265:14176) to increase the transmembrane transport of the motor protein therapeutic.

While not wishing to be bound by any particular theory, it is noted that hydrophilic polypeptides and organic molecules may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefore serve as an internalizing peptide for the subject motor protein therapeutic. Preferred growth factor-derived internalizing peptides include EGF (epidermal growth factor)-derived peptides, such as CMHIESLDSYTC (SEQ ID NO: 11) and CMYIEALDKYAC (SEQ ID NO: 12); TGF-beta (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Cellular internalization may also be mediated by ligands that bind to cell surface receptors for endocytosis. These ligands may include small molecules, such as a saccharide, steroid, or vitamin, peptides, such as TAT or EGF-derived peptides (as discussed above) or proteins such as antibodies or transferrin.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of motor protein therapeutics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

A particularly preferred pH-dependent membrane-binding internalizing peptide in this regard is aa1-aa2-aa3-EAALA(EALA)4-EALEALAA-amide (SEQ ID NO: 13), which represents a modification of the peptide sequence of Subbarao et al. (Biochemistry 26:2964, 1987). Within this peptide sequence, the first amino acid residue (aa1) is preferably a unique residue, such as cysteine or lysine, that facilitates chemical conjugation of the internalizing peptide to a targeting protein conjugate. Amino acid residues 2-3 may be selected to modulate the affinity of the internalizing peptide for different membranes. For instance, if both residues 2 and 3 are lys or arg, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Pore-forming proteins or peptides may also serve as internalizing peptides herein. Pore forming proteins or peptides may be obtained or derived from, for example, C9 complement protein, cytolytic T-cell molecules or NK-cell molecules. These moieties are capable of forming ring-like structures in membranes, thereby allowing transport of attached motor protein therapeutic through the membrane and into the cell interior.

In one embodiment, the motor protein therapeutic includes a transmembrane mediator to assist in subcellular trafficking. Examples may include a pH sensitive peptide for endosomal release such as the GALA peptide (Parente et al. 1988 J Biol Chem v263, 4724) or a fragment of the HA-2 protein (Wagner et al. 1992 PNAS v89, 7934), a small molecule for endosomal release, such as chloroquine, or peptides to mediate organelle entry. For example, several nuclear localization signals (NLSs) are known in the art, including the sequences from the SV40 virus, M9 peptide, and other proteins transported to the nucleus. For reviews on nuclear localization sequences, refer to Cartier et al. Gene Therapy 2002 v9:157 and Morris et al. Curr Opin Biotech 2000 v11:461.

G. Nucleic Acid Compositions

As described above, certain embodiments of the subject motor protein therapeutics feature peptides/polypeptides as both the motor protein binding moiety and the drug moiety. For those embodiments, another aspect of the invention provides expression vectors for expressing such entities. For instance, expression vectors are contemplated which include a nucleotide sequence encoding a polypeptide motor protein therapeutic, e.g., having at least one motor protein-binding sequence and peptide or polypeptide drug moiety which effects cellular function in a manner dependent upon its nuclear localization, which coding sequence is operably linked to at least one transcriptional regulatory sequence. Regulatory sequences for directing expression of the instant polypeptide motor protein therapeutics are art-recognized and are selected by a number of well understood criteria. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the polypeptide motor protein therapeutics of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

As will be apparent, the subject gene constructs can be used to cause expression of the subject polypeptide motor protein therapeutics in cells propagated in culture, e.g. to produce proteins or polypeptides, including polypeptide motor protein therapeutics, for purification.

This invention also pertains to a host cell transfected with a recombinant gene in order to express one of the subject polypeptides. The host cell may be any prokaryotic or eukaryotic cell. For example, a polypeptide motor protein therapeutics of the present invention may be expressed in bacterial cells such as E. coli, insect cells (baculovirus), yeast, or mammalian cells. Other suitable host cells are known to those skilled in the art.

Accordingly, the present invention further pertains to methods of producing the subject polypeptide motor protein therapeutics. For example, a host cell transfected with an expression vector encoding a protein of interest can be cultured under appropriate conditions to allow expression of the protein to occur. The protein may be secreted, by inclusion of a secretion signal sequence, and isolated from a mixture of cells and medium containing the protein. Alternatively, the protein may be retained cytoplasmically and the cells harvested, lysed and the protein isolated. A cell culture includes host cells, media and other byproducts. Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

Thus, a coding sequence for a polypeptide motor protein therapeutic of the present invention can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures.

Expression vehicles for production of a recombinant protein include plasmids and other vectors. For instance, suitable vectors for the expression of the instant polypeptide motor protein therapeutics include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide motor protein therapeutics by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the beta-gal containing pBlueBac III).

In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a polypeptide motor protein therapeutic of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86: 9079-9083; Julan et al., (1992) J. Gen Virol 73:3251-3255; and Goud et al., (1983) Virology 163: 251-254); or coupling cell surface ligands to the viral env proteins (Neda et al., (1991) J. Biol. Chem. 266: 14143-14146). Coupling can be in the form of the chemical cross-linking with a protein or other variety (e.g. lactose to convert the env protein to an asialoglycoprotein), as well as by generating polypeptide motor protein therapeutics (e.g. single-chain antibody/env polypeptide motor protein therapeutics). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6: 616; Rosenfeld et al., (1991) Science 252: 431-434; and Rosenfeld et al., (1992) Cell 68: 143-155). Suitable adenoviral vectors derived from the adenovirus strain Ad type 5 dl1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Ophthalmol Vis Sci 35:2662-2666) In addition to viral transfer methods, such as those illustrated above, non-viral methods can also be employed to cause expression of a protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding a motor protein-containing polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, any of the subject gene constructs can be used to transfect specific cells in vivo using a soluble polynucleotide carrier comprising an antibody conjugated to a polycation, e.g. poly-lysine (see U.S. Pat. No. 5,166,320). It will also be appreciated that effective delivery of the subject nucleic acid constructs via -mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art.

For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

H. Exemplary Formulations

The subject compositions may be used alone, or as part of a conjoint therapy with other pharmaceutical agents.

The motor protein therapeutics for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the motor protein therapeutics, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

Pharmaceutical formulations of the present invention can also include veterinary compositions, e.g., pharmaceutical preparations of the motor protein therapeutics suitable for veterinary uses, e.g., for the treatment of live stock or domestic animals, e.g., dogs.

Other formulations of the present invention include agricultural formulations, e.g., for application to plants.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a motor protein therapeutic at a particular target site.

The pharmaceutical compositions according to the present invention may be administered as either a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously. The pharmaceutical compositions of the present invention may be administered by any means that enables the motor protein moiety to reach the targeted cells. In some embodiments, routes of administration include those selected from the group consisting of oral, intravesically, intravenous, intraarterial, intraperitoneal, local administration into the blood supply of the organ in which the tumor resides or directly into the tumor itself. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, intravesically, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular motor protein therapeutic employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

Because the subject ligands are specifically targeted to tumor bladder cells, those modified motor protein peptides which comprise chemotherapeutics or toxins can be administered in doses less than those which are used when the chemotherapeutics or toxins are administered as unconjugated active agents, preferably in doses that contain up to 100 times less active agent. In some embodiments, modified motor protein peptides which comprise chemotherapeutics or toxins are administered in doses that contain 10-100 times less active agent as an active moiety than the dosage of chemotherapeutics or toxins administered as unconjugated active agents. To determine the appropriate dose, the amount of compound is preferably measured in moles instead of by weight. In that way, the variable weight of different modified motor protein peptides does not affect the calculation. Presuming a one to one ratio of modified motor protein peptide to active moiety in modified motor protein peptides of the invention, less moles of modified motor protein peptides may be administered as compared to the moles of unmodified motor protein peptides administered, preferably up to 100 times less moles.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with other antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

### EXAMPLES

Preparation of Dynein-Binding Peptide-Plasmid Conjugate

A dynein-binding peptide with a cysteine terminus (sequence: CSYSKETQPL, SEQ ID NO: 14) was synthesized by solid phase peptide synthesis and purified by reverse phase high pressure liquid chromatography. The peptide was conjugated to a rhodamine and maleimide double-labeled plasmid containing the GFP gene (plasmid purchased from Gene Therapy Systems, San Diego, Calif.) by mixing the peptide with DNA at 100:1 molar excess of peptide to DNA. Five minutes after mixing peptide with plasmid, TCEP (Tris(2-carboxyethyl)phosphine) was added to a final concentration of 5 mM to reduce dimerized peptides. The resulting solution was stirred at room temperature for 1 hour. A small aliquot was then collected, digested with Xmn I/BamH I for gel electrophoresis analysis. Approximately 50% of the plasmids were successfully conjugated. The free peptide in the remaining solution was removed by size exclusion chromatography through a G50 spin column.

Formulation with a Delivery System and Dynein-Mediated Transport to the Nuclear Surface in Cultured Cells.

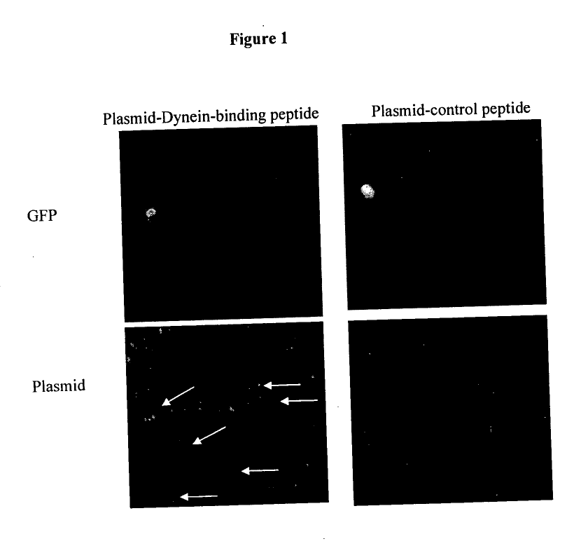

PC3 cells were plated at 100,000 cells/mL in 6-well plates containing a clean glass coverslip at the bottom of each well one day before transfection. For transfection, 1 μg of plasmid was mixed with a linear, cyclodextrin polycation (CD-IPEI) at 5+/− and mixed with cells in 1 mL of Opti-MEM. Transfection solution was removed 5 hours after exposure and replaced with complete media. Cells were washed with PBS, fixed with formalin (0.5 mL for 10 minutes), and washed 3 times with PBS. The coverslips were then removed from the wells and mounted on glass slides with Prolong Antifade (Molecular Probes, Eugene, Oreg.). Cells were visualized with a Olympus fluorescence microscope as shown in FIG. 1.

Equivalents

Those skilled in the art will recognize, or be able to ascertain using no more than routine experimentation, many equivalents to the specific embodiments of the invention described herein. Such equivalents are intended to be encompassed by the following claims.

All references, publications and patents cited in the specification above are herein incorporated by reference.

1. A motor protein therapeutic represented by one of the general formulas:
A-L-B (I)
or
A::B (II)
wherein
A represents a moiety which binds to a motor protein;
B represents a drug moiety which produces a change in the growth state or phenotype of a cell, or a change in concentrations or rates of protein internalization, processing, or excretion, in a manner dependent on or enhanced by a particular subcellular localization of B;
L represents a bond or a linker group which covalently attaches A and B, and
:: represents a non-covalent interaction between A and B.

2. The motor protein therapeutic of claim 1, wherein said motor protein is a conventional kinesin (kinesin I), a heterotrimeric kinesin II, a homodimeric kinesin II, an Unc104/KIF1 protein, or a myosin V.

3. The motor protein therapeutic of claim 1, wherein said motor protein is a dynein.

4. The motor protein therapeutic of claim 3, wherein said motor protein is a cytoplasmic dynein, and wherein said moiety A binds to a subunit of said cytoplasmic dynein.

5. The motor protein therapeutic of claim 4, wherein said subunit is a dynein light chain (DLC).

6. The motor protein therapeutic of claim 5, wherein said dynein light chain (DLC) is DLC8.

7. The motor protein therapeutic of claim 6, wherein said moiety A comprises a polypeptide or peptidomimetic with an amino acid sequence consisting essentially of SEQ ID NO: 1, or SEQ ID NO: 1.

8. The motor protein therapeutic of claim 6, wherein said moiety A comprises a polypeptide or peptidomimetic with an amino acid sequence consisting essentially of SEQ ID NOs: 2, 4, 5, 6, 7, or 8, or SEQ ID NOs: 2, 4, 5, 6, 7, or 8.

9. The motor protein therapeutic of claim 6, wherein said moiety A comprises a polypeptide or peptidomimetic with an amino acid sequence consisting essentially of Xaa1-Xaa2-Thr-Gln-Thr (SEQ ID NO: 3) or SEQ ID NO: 3,
wherein,
Xaa1 represents an amino acid residue with a positively charged sidechain,
Xaa2 represents an amino acid residue with a polar or a neutral sidechain.

10. The motor protein therapeutic of claim 9, wherein said Xaa1 is Lys, His or Arg.

11. The motor protein therapeutic of claim 9, wherein said Xaa2 is a polar sidechain amino acid selected from Arg, Asn, Asp, Cys, Glu, Gln, His, Lys, Ser or Thr.

12. The motor protein therapeutic of claim 9, wherein said Xaa2 is a neutral sidechain amino acid selected from Ala, Asn, Cys, Gln, Gly, His, Ile, Leu, Met, Phe, Pro, Ser, Thr, Trp, Tyr or Val.

13. The motor protein therapeutic of claim 12, wherein said Xaa2 is a small neutral sidechain amino acid selected from Gly, Ala or Ser.

14. The motor protein therapeutic of claim 11, wherein said Xaa2 is a negative polar sidechain amino acid selected from Asp or Glu.

15. The motor protein therapeutic of claim 9, wherein said Xaa2 is Ala, Gly, Glu or Ser.

16. The motor protein therapeutic of claim 6, wherein said moiety A is a DLC8-binding domain peptide sequence from neuronal nitric-oxide synthase (nNOS), proapoptotic Bcl-2 family protein Bim, Drosophilia mRNA localization protein Swallow, transcriptional regulator IκB, or postsynaptic scaffold protein GKAP.

17. The motor protein therapeutic of claim 16, wherein said moiety A is a DLC8-binding domain of nNOS (neuronal nitric-oxide synthase) represented by SEQ ID NO: 9.

18. The motor protein therapeutic of claim 6, wherein said moiety A is a small organic molecule which selectively binds to DLC8.

19. The motor protein therapeutic of claim 4, wherein said moiety A binds to cytoplasmic dynein with a dissociation constant K_{d} of no more than 10⁻⁴M.

20. The motor protein therapeutic of claim 1, wherein said moiety A comprises two or more repeats of a polypeptide which binds to said motor protein.

21. The motor protein therapeutic of claim 1, wherein said moiety A comprises a peptidomimetic of a polypeptide which binds to said motor protein.

22. The motor protein therapeutic of claim 1, wherein said moiety A comprises a small organic molecule.

23. The motor protein therapeutic of claim 1, wherein said drug moiety B is a nucleic acid.

24. The motor protein therapeutic of claim 23, wherein said nucleic acid is an oligonucleotide, an anti-sense oligonucleotide, an siRNA, or a plasmid.

25. The motor protein therapeutic of claim 1, wherein said drug moiety B is a polypeptide or a peptidomimetic thereof.

26. The motor protein therapeutic of claim 25, wherein said polypeptide is a transcriptional regulator, an inducer or inhibitor of programmed cell death, or an intrabody (functional antibody) with intracellular targets.

27. The motor protein therapeutic of claim 1, wherein said drug moiety B is a microsphere, a liposome, a small organic molecule, or a large synthetic molecule.

28. The motor protein therapeutic of claim 1, wherein said drug moiety B interacts with a nuclear target.

29. The motor protein therapeutic of claim 28, wherein said nuclear target is a transcription factor, a histone, or a protein or protein complex which interacts with DNA and regulate gene expression or chromatin structure, a nuclear hormone or steroid receptor, a histone acetylase or deacetylase, a DNA methyltransferase, an enzyme which covalently modifies DNA, a kinase, a phosphatase, a protease, a lipase, an RNA polymerase, a DNA polymerase, a DNA primase, a DNA topoisomerase, a DNA helicase, a nuclease, or an ATPase.

30. The motor protein therapeutic of claim 29, wherein said drug moiety B is an inhibitor or activator of said nuclear target.

31. The motor protein therapeutic of claim 1, wherein either said A or said B or both include functionalities for enhancing cellular uptake and/or transmembrane movement.

32. The motor protein therapeutic of claim 1, wherein either said A or said B or both includes groups which can be cleaved to form an active drug moiety B not linked to A.

33. The motor protein therapeutic of claim 1, wherein said subcellular localization is nucleus.

34. The motor protein therapeutic of claim 1, wherein said subcellular localization is lysosome, mitochondria, ER (Endoplasmic Reticulum), Golgi complex, or a membrane fraction.

35. The motor protein therapeutic of claim 1, wherein said A and said B are covalently linked by said L through chemical cross-linking.

36. The motor protein therapeutic of claim 1, wherein said L is an amino acid or a polypeptide.

37. The motor protein therapeutic of claim 35, wherein said L includes a bond that can be cleaved or enzymatically degraded under physiological condition once at said subcellular localization.

38. The motor protein therapeutic of claim 1, wherein said non-covalent interaction :: is ionic interaction, hydrogen bond, van der Waals interaction, hydrophobic interaction, or simultaneous binding of said A and B to a third molecule.

39. The motor protein therapeutic of claim 38, wherein said non-covalent interaction :: is direct binding between said A and said B.

40. The motor protein therapeutic of claim 39, wherein said direct binding between said A and said B is mediated by a pair of heterologous interacting polypeptides.

41. The motor protein therapeutic of claim 40, wherein said pair of heterologous interacting polypeptides are biotin and streptavidin or avidin.

42. The motor protein therapeutic of claim 38, wherein said non-covalent interaction becomes unstable under physiological condition once at said subcellular localization.

43. The motor protein therapeutic of claim 36, wherein said amino acid is Cysteine.

44. The motor protein therapeutic of claim 36, wherein said polypeptide comprises a terminal Cysteine and a spacer polypeptide.

45. The motor protein therapeutic of claim 44, wherein said spacer polypeptide comprises one or more repeats of Gly-Gly-Gly-Ser (SEQ ID NO: 15).

46. A nucleic acid encoding a proteinaceous motor protein therapeutic of claim 1, or the complement nucleic acid thereof.

47. A vector comprising the nucleic acid of claim 46.

48. A host cell comprising the vector of claim 47 or the nucleic acid of claim 46.

49. A method of delivering a drug moiety B to a particular subcellular localization of a cell, comprising contacting the cell with the motor protein therapeutic of claim 1.
